(19) United States
(12) Patent Application Publication
(10) Pub. No.: US 2026/0355244 A1
(43) Pub. Date: Nov. 12, 2026

(54) METHODS OF MANUFACTURING T CELLS

(71) Applicants: viTToria Biotherapeutics, Inc., Philadelphia, PA (US); THE TRUSTEES OF THE UNIVERSITY OF PENNSYLVANIA, Philadelphia, PA (US)

(72) Inventors: Nicholas A. Siciliano, Philadelphia, PA (US); Marco Ruella, Ardmore, PA (US)

(21) Appl. No.: 19/488,302

(22) PCT Filed: May 31, 2024
(86) PCT No.: PCT/US2024/031950

§ 371 Date: Nov. 26, 2025

Related U.S. Application Data
(60) Provisional application No. 63/505,265, filed on May 31, 2023.

(51) Int. Cl. A61K 35/17; A61K 40/11; A61K 40/31; A61P 35/00; C07K 16/28; C12N 5/00; C12N 5/0783; C12N 9/22; C12N 15/11; C12N 15/86; C12N 15/87

(52) U.S. Cl. CPC A61K 35/17; A61K 40/11; A61K 40/31; A61P 35/00; C07K 16/2896; C12N 5/0018; C12N 5/0636; C12N 9/226; C12N 15/111; C12N 15/86; C12N 15/87; A61K 2239/13; A61K 2239/48; C07K 2317/565; C12N 2310/20; C12N 2501/51; C12N 2501/515; C12N 2510/00; C12N 2740/15043

(57) ABSTRACT

Provided for herein is a method of manufacturing engineered T cells that can, for example comprise genetically editing unstimulated T cells prior to activation and expressing a chimeric antigen receptor.

### RELATED APPLICATIONS

This application claims the benefit of U.S. Provisional Application Ser. No. 63/505,265 filed May 31, 2023, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on May 23, 2024, is named “VTB-007WO_SL” and is 18,711 bytes in size.

### FIELD

The present embodiments relate to methods of producing genetically modified T cells, which can be used to produce modified T-cells, such as CAR-T cells, in a modified order of traditional steps and/or a shortened period of manufacturing, the product of which may be used, for example, to treat cancer or other immunological conditions.

### BACKGROUND

Gene editing technology and gene integration technology have expanded the ability to modify cells to contain heterologous nucleic acid molecules that can be used to express heterologous molecules, such as chimeric antigen receptors (CAR), nucleic acid molecules of interest, or proteins of interest. T cell lymphomas and leukemias have poor prognoses and there are few available treatments, but CAR T cell (CART) therapy has demonstrated efficacy in certain malignancies. CAR-T cells have traditionally been manufactured in a process that takes longer than seven days from cell harvest to infusion, and the step of activating the T cell population occurs prior to the genetic editing step. There is a need for a shortened manufacturing process, and by both shortening and modifying the order of genetic editing, fratricide in certain drug products can be avoided and a more robust population or populations of engineered T cells are able to be developed for successful infusion back into the patient for treatment of lymphoma and other cancers. The present embodiments satisfy these needs and others.

### BRIEF DESCRIPTION

In some embodiments, methods of preparing a T cell are provided, wherein the methods comprise genetically editing an unstimulated T cell. In some embodiments, the methods further comprise activating the genetically edited T cell. In some embodiments, the cell is genetically edited to modify a T cell genetic locus in addition to introducing a heterologous sequence. In some embodiments, the genetic locus is a CD5, CD2, or CD7 genetic locus.

In some embodiments, methods of manufacturing CAR-T cells are provided, wherein the method comprises the steps of: gene editing isolated unstimulated T cells; electroporating the T cells with a gene editing complex to genetically edit the T cell to produce gene edited T cells; activating the gene edited T-cells within 24 hours of electroporation by CD3/CD28 microbead antigen-induced activation; and transducing the genetically modified, activated T cells within 24 hours of activation with a lentivirus based vector comprising a heterologous nucleic acid molecule encoding a chimeric antigen receptor. In some embodiments, the methods further comprise culturing the transduced T cells for a period of time, such as 1-5 days. In some embodiments, the methods further comprise freezing (cryopreserving) the transduced T cells.

In some embodiments, methods of treating a cancer patient with a CAR-T cell are provided, the method comprising: gene editing isolated unstimulated T cells obtained from a subject; electroporating the T cells with a gene editing complex to genetically edit the T cell to produce gene edited T cells; activating the gene edited T-cells within 24 hours of electroporation by CD3/CD28 microbead antigen-induced activation; transducing the genetically modified, activated T cells within 24 hours of activation with a lentivirus based vector comprising a heterologous nucleic acid molecule encoding a chimeric antigen receptor; and administering the transduced T cells to the patient. In some embodiments, the subject is the cancer patient or the subject is a person who is not the patient (e.g., the cells are allogeneic to the patient).

### BRIEF DESCRIPTION OF DRAWINGS

FIG. 1: Illustrates a non-limiting embodiment of patient T cell harvesting, CART manufacturing process, and potential for infusion of the CART drug product back into the patient.

FIG. 2A-2C: Illustrate the assessment of CD5 KO CART5 cells generated via conventional and rapid manufacturing processes. FIG. 2A illustrates a comparison of CD5 expression. FIG. 2B illustrates a comparison of CAR5 expression. FIG. 2C illustrates a comparison of cell population doubling.

FIG. 3: Illustrates the difference in memory phenotypes between Mock, CD5 KO CART5 conventional, and CD5 KO CART5 rapid cells.

FIG. 4: Illustrates the difference in cytokine release between Mock, CD5 KO CART5 conventional, and CD5 KO CART5 rapid cells either in isolation (top panel) or after co-culture with Jurkat cells (bottom panel)

FIG. 5: Illustrates the difference in cytotoxicity against CD5+ Jurkat cells between Mock, CD5 KO CART5 conventional, and CD5 KO CART5 rapid cells at the given E:T ratios.

FIG. 6: Illustrates the effect of Mock, CD5 KO CART5 conventional, and CD5 KO CART5 rapid cells on tumor burden in an NSG mouse xenograft model.

FIG. 7: Illustrates the effect of Mock, CD5 KO CART5 conventional, and CD5 KO CART5 rapid cells on mouse survivability in an NSG mouse xenograft model.

FIG. 8: Illustrates the difference in hCD45+hCD3+ cells (i.e. CAR5 expressing cells) between Mock, CD5 KO CART5 conventional, and CD5 KO CART5 rapid cells in an NSG mouse xenograft model.

### DETAILED DESCRIPTION

Unless defined otherwise, all technical and scientific terms have the same meaning as is commonly understood by one of ordinary skill in the art to which the embodiments disclosed belongs. It is also to be understood that the terminology used herein is for the purpose of describing particular embodiments only, and is not intended to be limiting.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y,” the term “about” modifies both x and y and can be used interchangeably with the phrase “about x to about y” unless context dictates differently.

The term “signaling domain” refers to the functional portion of a protein which acts by transmitting information within the cell to regulate cellular activity via defined signaling pathways by generating second messengers or functioning as effectors by responding to such messengers.

“Activation,” as used herein in reference to a T cell, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically

The term “xenogeneic” refers to a graft derived from an animal of a different species.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, CD5rabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR comprises an antibody fragment. In some embodiments, the CAR comprises an antibody fragment that comprises a scFv.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. The term “antigen” can also refer to a molecule that an antibody or antibody-like molecule can bind to or is recognized by the antibody or antibody-like molecule. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present embodiments include, but are not limited to, the use of partial nucleotide sequences of more than one gene and that these nucleotide sequences can be arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

As used herein, to “alleviate” a disease means reducing the severity of one or more symptoms of the disease.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

As used herein, the term “individual” or “subject,” or “patient” used interchangeably, means any animal, including mammals, such as mice, rats, other rodents, rabbits, dogs, cats, swine, cattle, sheep, horses, or primates, such as humans. In some embodiments, the subject is a human. A subject that is “in need thereof” refers to a subject that has been identified as requiring treatment for the condition that is to be treated and is treated with the specific intent of treating such condition. The conditions can be, for example, any of the conditions described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “gene editing complex” can refer to a composition comprising, for example, a gRNA and a nuclease or a nuclease and any other factors that are necessary to facilitate the gene edit in the target cell. For example, if the nuclease is a CAS type nuclease, the gene editing complex can comprise a gRNA that targets the gene edit target and the CAS nuclease. Non-limiting examples of nucleases are provided for herein.

To “treat” a disease as the term is used herein, means to reduce the frequency or severity of at least one sign or symptom of a disease or disorder experienced by a subject. As used herein, “inhibit” or “treat” or “treatment” also includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The term “therapeutic” as used herein means a treatment and/or prophylaxis. A therapeutic effect is obtained by suppression, remission, or eradication of a disease state.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Parenteral” administration of an immunogenic composition includes, e.g., subcutaneous (s.c.), intravenous (i.v.), intramuscular (i.m.), or intrasternal injection, or infusion techniques.

The term “cancer” refers to a disease characterized by the uncontrolled growth of aberrant cells. Cancer cells can spread locally or through the bloodstream and lymphatic system to other parts of the body. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

In certain embodiments, the disease is a cancer. In addition, the compositions provided for herein can be used in methods for the treatment of any condition related to a cancer, such as a cell-mediated immune response against a tumor cell(s), where it is desirable to treat or alleviate the disease. The types of cancers to be treated include, but are not limited to, carcinoma, blastoma, sarcoma, certain leukemia or lymphoid malignancies, benign and malignant tumors, malignancies e.g., sarcomas, carcinomas, and melanomas. Other exemplary cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. In some embodiments, the cancer is the cancer is carcinoma, blastoma, sarcoma, leukemia, lymphoid malignancies, benign tumors, malignant tumors, sarcoma, carcinoma, melanoma, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, B cell related cancer or T cell related cancer. The cancers may be non-solid tumors (such as hematological tumors) or solid tumors. Adult tumors/cancers and pediatric tumors/cancers are also included. In one embodiment, the cancer is a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by the methods disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

As used herein, the phrase “in vivo” in reference to a cell being transduced, transfected or transformed in vivo, refers to a cell being transduced, transfected or transformed in the subject without the cells being removed from the subject before such cells are transduced, transfected or transformed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Non-limiting examples of lentiviruses are HIV, SIV, and FIV. Vectors or viral-like particles derived from lentiviruses can be used to transduce cells and deliver genes or other molecules and have them expressed in a cell either in vitro (ex-vivo) or in vivo. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “subject” includes living organisms, including those in which an immune response can be elicited (e.g., mammals). A “subject” or “patient,” as used therein, may be a human or non-human mammal. Non-human mammals include, for example, livestock and pets, such as ovine, bovine, porcine, canine, non-human primates, feline and murine mammals. In some embodiments, the subject is human.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

Ranges: throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

The term “knockdown” as used herein refers to a decrease in gene expression of one or more genes.

The term “knockout” as used herein refers to the ablation of gene expression of one or more genes.

A heterologous molecule of interest is meant to refer to any product that may be encoded by a nucleic acid molecule. As non-limiting examples, “cargo” or “heterologous molecule of interest” may refer to an siRNA, an shRNA, a peptide, a polypeptide, a protein, a viral payload, a viral genome, or a combination thereof. In some embodiments, the polypeptide is a chimeric antigen receptor (“CAR”). In some embodiments, the heterologous molecule of interest is an siRNA, an shRNA, a non-coding RNA (e.g. a guide RNA for a CRISPR system), a peptide, a polypeptide, a protein, a viral payload, a viral genome, a chimeric antigen receptor (“CAR”), or a combination thereof. In some embodiments, the heterologous molecule of interest is a CAR.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “unstimulated” when used in reference to a T cell refers to a T cell that has been isolated from a subject, such as cancer patient, that has not been activated, for example, with an exogenous factor, such as with one or more antibodies that bind to CD3 and/or CD28. Or, for example, an “unstimulated T cell” can refer to a T cell that has not been activated by the use of CD3/CD28 beads, a non-limiting example of which is described herein.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

The intracellular signaling domain can comprise the entire intracellular portion, or the entire native intracellular signaling domain, of the molecule from which it is derived, or a functional fragment or derivative thereof.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (δ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “Chimeric Antigen Receptor” or alternatively a “CAR” refers to a polypeptide, which when in an immune effector cell, provides the cell with specificity for a target cell, such as a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, domains are contiguous with each other in a single polypeptide or in multiple polypeptides. In some embodiments, the polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In some embodiments, the CARs comprise an extracellular domain (e.g., antigen binding domain) fused to CD3-zeta transmembrane and intracellular domain.

In some embodiments, the CAR comprises a target-specific binding element otherwise referred to as an antigen binding domain. The choice of antigen binding domain depends upon the type and number of ligands that define the surface of a target cell. For example, the antigen binding domain may be chosen to recognize a ligand that acts as a cell surface marker on target cells associated with a particular disease state (e.g. T cell lymphoma or leukemia).

In some embodiments, the CAR can be engineered to target a tumor antigen. The antigens discussed herein are merely included by way of example. The list is not intended to be exclusive and further examples will be readily apparent to those of skill in the art. Tumor antigens are proteins that are produced by tumor cells that elicit an immune response, particularly T-cell mediated immune responses. The selection of the antigen binding domain will depend on the particular type of cancer to be treated. Tumor antigens are well known in the art and include, for example, a glioma-associated antigen, carcinoembryonic antigen (CEA), (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The type of tumor antigen referred to in the disclosure may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as CD5, CD2, CD7, MART-1/MelanA (MART-I), gplOO (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Depending on the desired antigen to be targeted, the CAR can be engineered to include the appropriate antigen binding domain that is specific to the desired antigen target. For example, if CD5 is the desired antigen that is to be targeted, an antibody for CD5 can be used as the antigen binding domain for incorporation into the CAR. In certain embodiments, the antigen binding domain of the CAR targets CD5. In some embodiments, the antigen binding domain in the CAR is an anti-CD5 scFV. In some embodiments, the antigen binding domain is an anti-CD5 antibody.

In some embodiments, the CAR comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR include those associated with viral, bacterial and parasitic infections, autoimmune disease and cancer cells.

In certain embodiments, the antigen binding domain comprises a heavy chain variable region that comprises three heavy chain complementarity determining regions (HCDRs) and a light chain variable region that comprises three light chain complementarity determining regions (LCDRs).

In some embodiments, the CAR can be expressed in a T cell where the target on the surface of a T cell may include, but is not limited to CD2, CD3, CD4, CD5, CD7 or CD8. In some embodiments, the target is CD2. In some embodiments, the target is CD3. In some embodiments, the target is CD4. In some embodiments, the target is CD5. In some embodiments, the target is CD6. In some embodiments, the target is CD7. In some embodiments, the target is CD8.

In some embodiments, the CAR comprises an antigen binding domain against CD5. In some embodiments, the antigen binding domain against CD5 comprises a CD5 antibody, or a fragment thereof. In some embodiments, the antibody fragments are as provided for herein, such as but not limited to a scFv antibody, an antigen binding domain, an ankyrin repeat (e.g. DARPIN), a VHH domain antibody, a nanobody, single domain antibody, a FN3 domain, or any combination thereof. In some embodiments, the anti-CD5 antibody comprises a peptide selected from the following table, which illustrate the CDRs based on Kabat numbering:

Kabat CDRs

| Ab ID No | HCDR1 | HCDR2 | HCDR3 | LCDR1 | LCDR2 | LCDR3 |
|---|---|---|---|---|---|---|
| CD5AB1 | NYDVH | VIWNYGNT | NHGDGYYN | KASQDINK | YTSTLQP | LQYDNL |
| | (SEQ ID | DYNAAFIS | WYFDV | YIA (SEQ ID | (SEQ ID NO: | WT (SEQ |
| | NO: 1) | (SEQ ID NO: | (SEQ ID NO: | NO: 4) | 5) | ID NO: 6) |
| | | 2) | 3) | | | |

In some embodiments, the CAR comprises an antigen binding domain against CD5 comprising: (i) a light chain variable region comprising light chain CDR1, CDR2, and CDR3 sequences, wherein the light chain CDR1 sequence has the amino acid sequence of SEQ ID NO: 4, the light chain CDR2 sequence has the amino acid sequence of SEQ ID NO: 5, and the light chain CDR3 sequence has the amino acid sequence of SEQ ID NO: 6; and (ii) a heavy chain variable region comprising heavy chain CDR1, CDR2, and CDR3 sequences, wherein the heavy chain CDR1 sequence has the amino acid sequence of SEQ ID NO: 1, the heavy chain CDR2 sequence has the amino acid sequence of SEQ ID NO: 2, and the heavy chain CDR3 sequence has the amino acid sequence of SEQ ID NO: 3.

Although the preceding paragraphs may make reference to CDRs under the Kabat system the equivalent CDR sequences can be used from the IMGT and CHOTHIA designations.

In some embodiments, the CAR comprises an antigen binding domain against CD5 comprising a heavy chain variable region peptide having a sequence as provided in the table below:

| SEQ ID NO: | AB ID NO: | Sequence |
|---|---|---|
| 7 | CD5AB1 | EVQLVESGPGLVQPSQSLSITCTVSGFSLTNYDVHWVRQSPGKG |
| | | LEWLGVIWNYGNTDYNAAFISRLSIRKDSSKSQVFFTMSSLQTP |
| | | DTAIYYCARNHGDGYYNWYFDVWGTGTTVTVSS |

In some embodiments, the CAR comprises an antigen binding domain against CD5 comprising a light chain variable region peptide having a sequence as provided in the table below:

| SEQ ID NO: | AB ID NO: | Sequence |
|---|---|---|
| 8 | CD5AB1 | NIVLTQSPSSLSESLGGKVTITCKASQDINKYIAWYQYKPGKGPR |
| | | LLIHYTSTLQPGIPSRFSGSGSGRDYSFSISNLEPEDIATYYCLQYD |
| | | NLWTFGGGTKLEIK |

In some embodiments, the CAR comprises an antigen binding domain against CD5 comprising a V_{H} peptide of SEQ ID NO: 7. In some embodiments, the CAR comprises an antigen binding domain against CD5 comprising a V_{L} peptide of SEQ ID NO: 8. In some embodiments, the CAR comprises an antigen binding domain against CD5 comprising a V_{H} peptide and a V_{L} peptide, wherein the V_{H} peptide comprises an amino acid sequence of SEQ ID NO: 7 and the V_{L} peptide comprises an amino acid sequence of SEQ ID NO: 8. In some embodiments, the CAR comprises an antigen binding domain against CD5 comprising a V_{H} peptide and a V_{L} peptide, wherein the V_{H} peptide comprises an amino acid sequence having at least 90% identity to an amino acid sequence of SEQ ID NO: 7 and the V_{L} peptide comprises an amino acid sequence an amino acid sequence having at least 90% identity to an amino acid sequence of SEQ ID NO: 8. In some embodiments, the CAR comprises an antigen binding domain against CD5 comprising a V_{H} peptide and a V_{L} peptide, wherein the V_{H} peptide comprises an amino acid sequence having at least 90% identity to an amino acid sequence of SEQ ID NO: 7, provided that the V_{H} comprises a HCDR1 comprising an amino acid sequence of SEQ ID NO: 1, a HCDR2 comprising an amino acid sequence of SEQ ID NO: 2, and a HCDR3 comprising an amino acid sequence of SEQ ID NO: 3; and the V_{L} peptide comprises an amino acid sequence an amino acid sequence having at least 90% identity to an amino acid sequence of SEQ ID NO: 8, provided that the V_{L} comprises a LCDR1 comprising an amino acid sequence of SEQ ID NO: 4, a LCDR2 comprising an amino acid sequence of SEQ ID NO: 5, and a LCDR3 comprising an amino acid sequence of SEQ ID NO: 6.

The V_{H} and the V_{L} sequences can be in any format, including, but not limited to an scFv format where the V_{H} and V_{L} regions are linked with a peptide linker. Non-limiting examples of peptide linkers that can be used to link various peptides include, but are not limited to, GGGGS (SEQ ID NO: 13), GGGGSGGGGS (SEQ ID NO: 14), (GGGGS)₃ (SEQ ID NO: 15), (GGGGS)₄ (SEQ ID NO: 16), (GGGS)ₙ, wherein n is and integer selected from 1, 2, 3, 4, 5, 6, 7, 8, 9, or 10 (SEQ ID NO: 17), or GGGS (SEQ ID NO: 18). Additional peptide linkers are known to the skilled artisan, and any such peptide linker is within the scope of the present disclosure.

In some embodiments, the CAR comprises an antigen binding domain against CD5 comprising an amino acid sequence having at least 85%, 86%, 87%, 88%, 89%, 90%, 91%, 92%, 93%, 94%, 95%, 96%, 97%, 98%, or 99% identity to an amino acid sequence of SEQ ID NO: 9:

(SEQ ID NO: 9)
EVQLVESGPGLVQPSQSLSITCTVSGFSLTNYDVHWVRQSPGKGLEWL
GVIWNYGNTDYNAAFISRLSIRKDSSKSQVFFTMSSLQTPDTAIYYCAR
NHGDGYYNWYFDVWGTGTTVTVSSGGGGSGGGGSGGGGSNIVLTQS
PSSLSESLGGKVTITCKASQDINKYIAWYQYKPGKGPRLLIHYTSTLQP
GIPSRFSGSGSGRDYSFSISNLEPEDIATYYCLQYDNLWTFGGGTKLEIK

In some embodiments, the CAR comprises an antigen binding domain against CD5 comprising an amino acid sequence having at least 90% identity to an amino acid sequence of SEQ ID NO: 9. In some embodiments, the CAR comprises an antigen binding domain against CD5 comprising an amino acid sequence having at least 95% identity to an amino acid sequence of SEQ ID NO: 9. In some embodiments, the CAR comprises an antigen binding domain against CD5 comprising an amino acid sequence having at least 98% identity to an amino acid sequence of SEQ ID NO: 9. In some embodiments, the CAR comprises an antigen binding domain against CD5 comprising an amino acid sequence of SEQ ID NO: 9.

In some embodiments, the CAR comprises an antigen binding domain against CD5 comprising an amino acid sequence having at least 85%, 86%, 87%, 88%, 89%, 90%, 91%, 92%, 93%, 94%, 95%, 96%, 97%, 98%, or 99% identity to an amino acid sequence of SEQ ID NO: 10:

(SEQ ID NO: 10)
NIVLTQSPSSLSESLGGKVTITCKASQDINKYIAWYQYKPGKGPRLLIH
YTSTLQPGIPSRFSGSGSGRDYSFSISNLEPEDIATYYCLQYDNLWTFGG
GTKLEIKGGGGSGGGGSGGGGSEVQLVESGPGLVQPSQSLSITCTVSG
FSLTNYDVHWVRQSPGKGLEWLGVIWNYGNTDYNAAFISRLSIRKDS
SKSQVFFTMSSLQTPDTAIYYCARNHGDGYYNWYFDVWGTGTTVTV
SS

In some embodiments, the CAR comprises an antigen binding domain against CD5 comprising an amino acid sequence having at least 90% identity to an amino acid sequence of SEQ ID NO: 10. In some embodiments, the CAR comprises an antigen binding domain against CD5 comprising an amino acid sequence having at least 95% identity to an amino acid sequence of SEQ ID NO: 10. In some embodiments, the CAR comprises an antigen binding domain against CD5 comprising an amino acid sequence having at least 98% identity to an amino acid sequence of SEQ ID NO: 10. In some embodiments, the CAR comprises an antigen binding domain against CD5 comprising an amino acid sequence of SEQ ID NO: 10.

In some embodiments, the CAR comprising an antigen binding domain against CD5 comprises an amino acid sequence that is at least 85%, 86%, 87%, 88%, 89%, 90%, 91%, 92%, 93%, 94%, 95%, 96%, 97%, 98%, 99%, or 100% identical to a sequence of SEQ ID NO: 11:

(SEQ ID NO: 11)
MALPVTALLLPLALLLHAARPGSEVQLVESGPGLVQPSQSLSITCTVSGF
SLTNYDVHWVRQSPGKGLEWLGVIWNYGNTDYNAAFISRLSIRKDSSKSQ
VFFTMSSLQTPDTAIYYCARNHGDGYYNWYFDVWGTGTTVTVSSGGGGSG
GGGSGGGGSNIVLTQSPSSLSESLGGKVTITCKASQDINKYIAWYQYKPG
KGPRLLIHYTSTLQPGIPSRFSGSGSGRDYSFSISNLEPEDIATYYCLQY
DNLWTFGGGTKLEIKSRITTPAPRPPTPAPTIASQPLSLRPEACRPAAGG
AVHTRGLDFACDIYIWAPLAGTCGVLLLSLVITLYCHMKRGRKKLLYIFK
QPFMRPVQTTQEEDGCSCRFPEEEEGGCELTSRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR

In some embodiments, the CAR comprising an antigen binding domain against CD5 comprises an amino acid sequence that is at least 90% identical to a sequence of SEQ ID NO: 11. In some embodiments, the CAR comprising an antigen binding domain against CD5 comprises an amino acid sequence that is at least 95% identical to a sequence of SEQ ID NO: 11. In some embodiments, the CAR comprising an antigen binding domain against CD5 comprises an amino acid sequence that is at least 98% identical to a sequence of SEQ ID NO: 11. In some embodiments, the CAR comprising an antigen binding domain against CD5 comprises an amino acid sequence of SEQ ID NO: 11.

In some embodiments, the CAR comprising an antigen binding domain against CD5 comprises an amino acid sequence that is at least 85%, 86%, 87%, 88%, 89%, 90%, 91%, 92%, 93%, 94%, 95%, 96%, 97%, 98%, 99%, or 100% identical to a sequence of SEQ ID NO: 12:

(SEQ ID NO: 12)
EVQLVESGPGLVQPSQSLSITCTVSGFSLTNYDVHWVRQSPGKGLEWLGV
IWNYGNTDYNAAFISRLSIRKDSSKSQVFFTMSSLQTPDTAIYYCARNHG
DGYYNWYFDVWGTGTTVTVSSGGGGSGGGGSGGGGSNIVLTQSPSSLSES
LGGKVTITCKASQDINKYIAWYQYKPGKGPRLLIHYTSTLQPGIPSRFSG
SGSGRDYSFSISNLEPEDIATYYCLQYDNLWTFGGGTKLEIKSRTTTPAP
RPPTPAPTIASQPLSLRPEACRPAAGGAVHTRGLDFACDIYIWAPLAGTC
CHMKRGRKKLLYIFKQPFMRPVQTTQEEDGCSCRFPEEEEGGCELTSRVK
GVLLLSLVITLYFSRSADAPAYQQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR

In some embodiments, the CAR comprising an antigen binding domain against CD5 comprises an amino acid sequence that is at least 90% identical to a sequence of SEQ ID NO: 12. In some embodiments, the CAR comprising an antigen binding domain against CD5 comprises an amino acid sequence that is at least 95% identical to a sequence of SEQ ID NO: 12. In some embodiments, the CAR comprising an antigen binding domain against CD5 comprises an amino acid sequence that is at least 98% identical to a sequence of SEQ ID NO: 12. In some embodiments, the CAR comprising an antigen binding domain against CD5 comprises an amino acid sequence of SEQ ID NO: 12.

The embodiments provided for herein for a CAR comprising an antigen binding domain against CD5 are exemplary only and are not meant to be limiting in any way. Additional CAR constructs, CD5 or otherwise, that are suitable for use with the present disclosure may be found, for example, in International Patent Publication WO 2020/132327, U.S. Pat. No. 11,673,964, and U.S. Patent Application Publication No. 2023/0331864, each of which are incorporated by reference herein in their entirety.

In some embodiments, methods of delivering a cargo of interest to a cell in a subject are provided. In some embodiments, the cargo is a chimeric antigen receptor or as otherwise provided for herein.

Also provided herein are methods of treating a disease in a subject in need thereof.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In some embodiments, a cancer associated with expression of a tumor antigen as described herein is a hematological cancer. In some embodiments, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiments, the tumor antigen-expressing cells produced detectable levels of a tumor antigen protein at one point, and subsequently produced substantially no detectable tumor antigen protein.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

In some embodiments, the subject has been treated with a therapeutic agent targeting the disease or condition, e.g. the tumor, prior to infusion of a CAR-T cell. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the composition is administered prophylactically, e.g., to reduce the likelihood of or prevent relapse. In some aspects, the subject has not received prior treatment with another therapeutic agent.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat, or rabbit, having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

“Tumor antigen” or “hyperproliferative disorder antigen” or “antigen associated with a hyperproliferative disorder” refers to antigens that are common to specific hyperproliferative disorders. In some embodiments, the hyperproliferative disorder antigens are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample.

“Refractory” as used herein refers to a disease, e.g., cancer, that does not respond to a treatment. In some embodiments, a refractory cancer can be resistant to a treatment before or at the beginning of the treatment. In other embodiments, the refractory cancer can become resistant during a treatment. A refractory cancer is also called a resistant cancer.

“Relapsed” as used herein refers to the return of a disease (e.g., cancer) or the signs and symptoms of a disease such as cancer after a period of improvement, e.g., after prior treatment of a therapy, e.g., cancer therapy

“CD5” as the term is used herein, refers to the gene, and the protein encoded by said gene, of cluster of differentiation 5, CD5 can also be known as T1 or LEU1. In the human genome, CD5 is located on chromosome 11, with the GenBank number NC_00011.10.

A “system” as the term is used herein in connection with gene editing or CD5 inhibition, refers to a group of molecules, e.g., one or more molecules, which together act to effect a desired function.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

A “binding partner” as the term is used herein in the context of a CD5 binding partner, refers to a molecule, e.g., a protein, which interacts, e.g., binds to, CD5 protein.

A “dominant negative” gene product or protein is one that interferes with the function of another gene product or protein. The other gene product affected can be the same or different from the dominant negative protein. Dominant negative gene products can be of many forms, including truncations, full length proteins with point mutations or fragments thereof, or fusions of full length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions. In some embodiments, a dominant negative CD5 is a catalytically inactive CD5.

The administration of the drug product may be carried out in any convenient manner known to those of skill in the art. For example, the drug product may be administered to a subject by aerosol inhalation, injection, ingestion, transfusion, implantation or transplantation. The drug product described herein may be administered to a patient transarterially, subcutaneously, intradermally, intratumorally, intranodally, intramedullary, intramuscularly, by intravenous (i.v.) injection, intraperitoneally, intranasally, intracranially, or intraosseously. In other instances, the drug product is injected directly into a site of a local disease site in the subject, a lymph node, an organ, a tumor, and the like. In some instances, the drug product is delivered by infusion into the patient.

In certain embodiments, the subject is provided a secondary treatment. Secondary treatments include but are not limited to chemotherapy, radiation, surgery, and medications. In some embodiments, the subject is not provided a secondary treatment.

In some embodiments, the methods are performed without a lymphodepletion step, such as the administration of cyclophosphamide and/or fludarabine.

In some embodiments, the subject can be administered a conditioning therapy after the administration of the compositions to kill certain immune cells that are not transduced with the CAR encoded by the compositions. This can be done by including a selection marker that is encoded by the nucleic acid cargo of interest. In some embodiments, the conditioning therapy comprises administering an effective amount of cyclophosphamide to the subject. In some embodiments, the conditioning therapy comprises administering an effective amount of fludarabine to the subject. In some embodiments, the conditioning therapy comprises administering an effective amount of a combination of cyclophosphamide and fludarabine to the subject.

In some embodiments, a specific dosage regimen of the present disclosure includes a lymphodepletion step after the administration of the composition. In an exemplary embodiment, the lymphodepletion step includes administration of cyclophosphamide and/or fludarabine.

In some embodiments, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day). In an exemplary embodiment, the dose of cyclophosphamide is about 300 mg/m2/day. In some embodiments, the lymphodepletion step includes administration of fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the dose of fludarabine is about 30 mg/m2/day.

In some embodiments, the lymphodepletion step includes administration of cyclophosphamide at a dose of between about 200 mg/m2/day and about 2000 mg/m2/day (e.g., 200 mg/m2/day, 300 mg/m2/day, or 500 mg/m2/day), and fludarabine at a dose of between about 20 mg/m2/day and about 900 mg/m2/day (e.g., 20 mg/m2/day, 25 mg/m2/day, 30 mg/m2/day, or 60 mg/m2/day). In an exemplary embodiment, the lymphodepletion step includes administration of cyclophosphamide at a dose of about 300 mg/m2/day, and fludarabine at a dose of about 30 mg/m2/day.

It is known in the art that one of the adverse effects of the use of CAR T cells can be the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more SCRS.

Accordingly, in some embodiments, the methods comprise, following the diagnosis of CRS, appropriate CRS management strategies to mitigate the physiological symptoms of uncontrolled inflammation without dampening the antitumor efficacy of the in vivo generated cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In certain embodiments, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80 °C at a rate of 1° per minute and stored in the vapor phase of a liquid nitrogen storage tank. Other methods of controlled freezing may be used as well as uncontrolled freezing immediately at −20 °C or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

Naturally-occurring CRISPR/Cas systems are found in approximately 40% of sequenced eubacteria genomes and 90% of sequenced archaea. Grissa et al. (2007) BMC Bioinformatics 8:172. This system is a type of prokaryotic immune system that confers resistance to foreign genetic elements such as plasmids and phages and provides a form of acquired immunity. Barrangou et al. (2007) Science 315:1709-1712; Marragini et al. (2008) Science 322:1843-1845.

The CRISPR/Cas system has been modified for use in gene editing (silencing, enhancing or changing specific genes) in eukaryotes such as mice or primates. Wiedenheft et al. (2012) Nature 482:331-8. This is accomplished by, for example, introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in an exemplary CD5 CRISPR/Cas system, the spacers are derived from the CD5 gene sequence, or a sequence of its regulatory elements.

RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327:167-170; Makarova et al. (2006) Biology Direct 1:7. The spacers thus serve as templates for RNA molecules, analogously to siRNAs. Pennisi (2013) Science 341:833-836.

As these naturally occur in many different types of bacteria, the exact arrangements of the CRISPR and structure, function and number of Cas genes and their product differ somewhat from species to species. Haft et al. (2005) PLOS Comput. Biol. 1: e60; Kunin et al. (2007) Genome Biol. 8: R61; Mojica et al. (2005) J. Mol. Evol. 60:174-182; Bolotin et al. (2005) Microbiol. 151:2551-2561; Pourcel et al. (2005) Microbiol. 151:653-663; and Stern et al. (2010) Trends. Genet. 28:335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321:960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341:833-836.

The CRISPR/Cas system can thus be used to modify, e.g., delete one or more nucleic acids, the CD5 gene, or a CD5 gene regulatory element, or introduce a premature stop which thus decreases expression of a functional CD5 and this can be done to insert the heterologous nucleic acid molecule at a CD gene locus, which includes the CD5 gene regulatory elements (e.g., promoters, enhancers, and the like). The CRISPR/Cas system can alternatively be used like RNA interference, turning off the CD5 gene in a reversible fashion and/or insert the heterologous nucleic acid molecule in a CD5 gene locus if the insertion doesn't inhibit the expression of the CD5 gene.

CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization.

Artificial CRISPR/Cas systems can be generated to target insertion at the CD5 gene locus using technology known in the art, e.g., that are described in U.S. Publication No. 20140068797, WO2015/048577, and Cong (2013) Science 339:819-823. Other artificial CRISPR/Cas systems that are known in the art may also be generated e.g., which are described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated to insert the heterologous gene of interest, for example, engineering a CRISPR/Cas system to include a gRNA molecule comprising a targeting sequence that hybridizes to a sequence of a CD5 gene. In some embodiments, the gRNA comprises a targeting sequence which is fully complementarity to 15-25 nucleotides, e.g., 20 nucleotides, of a CD5 gene. In some embodiments, the 15-25 nucleotides, e.g., 20 nucleotides, of a CD5 gene, are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C).

Thus, in some embodiments, the heterologous nucleic acid molecule can be introduced into the cell along with the CRISPR/Cas system, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the CRISPR/Cas system. As shown herein, but without being bound by theory, such integration may lead to the expression of the CAR as well as disruption of the CD5 gene. Such foreign DNA molecule is referred to herein as “template DNA.” In some embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

In some embodiments, the CRISPR/Cas system comprises Cas9, e.g., S. pyogenes Cas9, and a gRNA comprising a targeting sequence which hybridizes to a sequence of the CD5 gene. In some embodiments, the CRISPR/Cas system comprises nucleic acid encoding a CD5 gRNA and nucleic acid encoding a Cas protein, e.g., Cas9, e.g., S. pyogenes Cas9. In some embodiments, the CRISPR/Cas system comprises a CD5 gRNA and nucleic acid encoding a Cas protein, e.g., Cas9, e.g., S. pyogenes Cas9.

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29:135-6; and Boch et al. (2009) Science 326:1509-12; Moscou et al. (2009) Science 326:3501.

TALENs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is, for example, a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29:143-8; Hockemeyer et al. (2011) Nature Biotech. 29:731-734; Wood et al. (2011) Science 333:307; Doyon et al. (2010) Nature Methods 8:74-79; Szczepek et al. (2007) Nature Biotech. 25:786-793; and Guo et al. (2010) J. Mol. Biol. 200:96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29:143-8.

A CD5 TALEN can be used inside a cell to produce a double-stranded break (DSB). A mutation and insertion can be introduced at the break site if the repair mechanisms improperly repair the break via non-homologous end joining. For example, improper repair may introduce a frame shift mutation. Alternatively, foreign DNA, such as the heterologous nucleic acid molecule can be introduced into the cell along with the TALEN, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the TALEN. As shown herein, in the examples, but without being bound by theory, such integration may lead to the expression of the CAR as well as disruption of the CD5 gene. Such foreign DNA molecule is referred to herein as “template DNA.” In some embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

TALENs specific to sequences in CD5 can be constructed using any method known in the art, including various schemes using modular components. Zhang et al. (2011) Nature Biotech. 29:149-53; Geibler et al. (2011) PLOS ONE 6: e19509; U.S. Pat. Nos. 8,420,782; 8,470,973, the contents of which are hereby incorporated by reference in their entirety.

“ZFN” or “Zinc Finger Nuclease” refer to a zinc finger nuclease, an artificial nuclease which can be used to modify, e.g., delete one or more nucleic acids of, a desired nucleic acid sequence.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188:773-782; and Kim et al. (1996) Proc. Natl. Acad. Sci. USA 93:1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95:10570-5.

Also like a TALEN, a ZFN can create a double-stranded break in the DNA, which can create a frame-shift mutation if improperly repaired, leading to a decrease in the expression and amount of CD5 in a cell. ZFNs can also be used with homologous recombination to mutate the CD5 gene, or to introduce the heterologous nucleic acid molecule, such as one, encoding a CAR, at a site at or near the targeted sequence. As discussed above, the nucleic acid encoding a CAR may be introduced as part of a template DNA. In some embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

ZFNs specific to sequences in the CD5 gene can be constructed using any method known in the art. See, e.g., Provasi (2011) Nature Med. 18:807-815; Torikai (2013) Blood 122:1341-1349; Cathomen et al. (2008) Mol. Ther. 16:1200-7; and Guo et al. (2010) J. Mol. Biol. 400:96; U.S. Patent Publication 2011/0158957; and U.S. Patent Publication 2012/0060230, the contents of which are hereby incorporated by reference in their entirety. In some embodiments, the ZEN gene editing system may also comprise nucleic acid encoding one or more components of the ZEN gene editing system, e.g., a ZEN gene editing system targeted to CD5.

Without being bound by theory, it is believed that use of gene editing systems (e.g., CRISPR/Cas gene editing systems) which target CD5, may allow one to inhibit one or more functions of CD5, by, for example, causing an editing event which results in expression of a truncated CD5. Again, without being bound by theory, such truncated CD5 proteins may preserve one or more functions of the CD5 (e.g., a scaffolding function), while inhibiting one or more other functions of the CD5 (e.g., a catalytic function), and as such, may be preferable. Gene editing systems which target a late exon or intron of a CD5 gene, may be particularly preferred in this regard. In some embodiments, the gene editing system CD5 inhibitor targets a late exon or intron of the CD5 gene.

Without being bound by theory, it may also be preferable in other embodiments to target an early exon or intron of CD5 gene, for example, to introduce a premature stop codon in the targeted gene which results in no expression of the gene product, or expression of a completely non-functional gene product. Gene editing systems which target an early exon or intron of a CD5 gene, may be particularly preferred in this regard. In some embodiments, the gene editing system CD5 inhibitor targets an early exon or intron of the CD5 gene.

Certain embodiments involve the use of electroporation to facilitate entry of one or more nucleic acid molecules into host cells.

As used herein, “electroporation” or “electrocharging” refers to the application of an electrical current or electrical field in a cell to facilitate entry of a nucleic acid molecule into the cell. Those skilled in the art will understand that any electroporation method, device, and technique can be used. For example, flow electroporation can be performed using Lonza, MaxCyte STX®, MaxCyte VLX®, or MaxCyte GT® flow electroporation instruments. In specific embodiments, static or flow electroporation is used with parameters indicated throughout. Other non-limiting methods, devices, and systems are described in WO2005113820, WO2003018751, WO2003018751, WO2010009252, WO2021231497, WO2006060409, WO2020086701, WO2022232802, WO/2004/031353, WO/2007/021993, or WO/2007/021994, each of which is hereby incorporated by reference in its entirety.

The transfecting cells by electroporation, such as by flow electroporation, can achieve transfection efficiencies greater than 40%, greater than 50%, and greater than 60%, 70%, 80%, or 90% (or any range derivable from the themselves). Transfection efficiency can be measured as the percentage of cells expressing the product of the gene or the level of secretion of the product expressed by the gene. The cells maintain high viability during and after the electroporation procedure. Viability is can be greater than 50%. The viability of electroporated cells can be at most or at least about 5%, 10%, 15%, 20%, 25%, 30%, 35%, 40%, 45%, 50%, 55%, 60%. %, 65%, 70%, 75%, 80%, 85%, 90% or 95% (or any interval derivable thereof), of the viability of the initial non-electroporated population or of a population electroporated and transfected with a control construct.

“Electroporation” may be interchangeably used with “electrotransfection” and “electroloading” with emphasis on general meaning of this technology, the transgene expression and the transference of molecules into cytoplasm, respectively. Those of skill in the art are familiar with methods of electroporation. The electroporation may be, for example, flow electroporation or static electroporation. In some embodiments, the method of transfecting the cells comprises use of an electroporation device as described in U.S. patent application Ser. No. 10/225,446, incorporated herein by reference. Methods and devices for electroporation are also described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, each of which are incorporated by reference in its entirety.

Other methods and devices that can be used to electroporate cells are described in U.S. patent application Ser. No. 10/225,446, filed Aug. 21, 2002, which is the entire disclosure of which is specifically incorporated herein by reference.

In some embodiments, electroloading may be carried out as described in U.S. Pat. No. 5,612,207 (specifically incorporated herein by reference), U.S. Pat. No. 5,720,921 (specifically incorporated herein by reference), U.S. Pat. No. 6,074,605 (specifically incorporated herein by reference); U.S. Pat. No. 6,090,617 (specifically incorporated herein by reference); and U.S. Pat. No. 6,485,961 (specifically incorporated herein by reference). Other methods and devices for electroloading that may be used are as described in, for example, published PCT Application Nos. WO 03/018751 and WO 2004/031353; U.S. patent application Ser. Nos. 10/781,440, 10/080,272, and 10/675,592; and U.S. Pat. Nos. 6,773,669, 6,090,617, 6,617,154, all of which are incorporated by reference.

In some embodiments, the cell that is electroporated is an immune cell, such as an immune effector cell or T cells.

In certain embodiments, the transfection method used is electroporation. In a further embodiment, the method of electroporation is flow electroporation. Flow electroporation, which refers to a method comprising: transferring a suspension of cells and loading molecules into an apparatus comprising a fluid chamber or fluid flow path; wherein said fluid chamber or fluid flow path comprises electrodes disposed along the sides of the fluid chamber or fluid flow path and configured to subject biological particles within the fluid chamber or path of fluid flow to an electric field suitable for electroporation, and for transferring the electroporated cell suspension to the outside of the apparatus. Such a method is particularly effective for a large scale volume of cells. Static electroporation, in contrast, involves the electroporation of a fixed and limited volume of cells due to the restrictions associated with the movement of electricity through a liquid and the distance between opposing electrodes.

In certain embodiments, the T cells disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand. The cells can also be expanded in the presence of anti-CD3 and/or CD28 antibodies. These can be contacted with the cells in the form of a beads, which are known in the art.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example, 1, 2, 3, 4, 5, 6, 7, 8, 9, 10, 11, 12, 13, 14, or more days. In some embodiments, the cells are cultured for no more than 5 days or as described herein.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In some embodiments, the cells may be cultured for several hours (about 3 hours) to about 14 days or any hourly integer value in between, including after activation. Conditions appropriate for T cell culture include an appropriate media (e.g., Minimal Essential Media or RPMI Media 1640 or, X-vivo 15, (Lonza) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37 °C) and atmosphere (e.g., air plus 5% CO2).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

In some embodiments, methods of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

In some embodiments, methods of preparing an engineered cell, such as a T cell comprises genetically modifying the T cell, such as an unstimulated T cell and activating the genetically modified T cell.

In some embodiments, method of preparing an engineered cell, such as a T cell, comprises genetically editing a naïve or un-activated (unstimulated) T cell in vivo or in ex vivo.

In some embodiments, the T cell comprises a heterologous nucleic acid molecule, which can be introduced into the T cell through a lentiviral vector.

In some embodiments, the cell is an immune cell, such as those provided for herein, which includes, but is not limited to a T-cell, NK cell, or a B-cell. In some embodiments, the T cell is a αβ T cell. In some embodiments, the B-cell is a B1 B-cell. In some embodiments, the cell is a T cell.

In some embodiments the cell expresses CD5 or expresses a functional CD5 from the native CD5 gene locus. In some embodiments the cell comprising the heterologous nucleic acid molecule does not express CD5 or express a functional CD5 from the native CD5 gene locus.

In some embodiments, the heterologous nucleic acid molecule encodes for a protein of interest. In some embodiments, the heterologous nucleic acid molecule encodes for a chimeric antigen receptor. Non-limiting examples of chimeric antigen receptors are provided for herein. In some embodiments, the heterologous nucleic acid molecule encodes for an antibody, chemokine, hormone, cytokine, and the like. In some embodiments, the heterologous nucleic acid molecule encodes for an RNA molecule, such as a miRNA, siRNA, mRNA, antisense molecule, and the like.

In some embodiments, the method of preparing an engineered T cell comprises the step of activating the T cell. In some embodiments, the method of preparing an engineered T cell comprises the steps of genetically editing the T cell and activating the T cell, wherein the step of genetically editing the T cell occurs prior to activation of the T cell. In some embodiments, the method further comprises harvesting the engineered T cell. In some embodiments, the engineered T cells are harvested no later than 7 days after activation. In some embodiments, the engineered T cells are harvested between 1 and 7 days after activation. In some embodiments, the engineered T cells are harvested 1, 2, 3, 4, 5, 6, or 7 days after activation.

In some embodiments, the engineered T cells are harvested 1 day after activation. In some embodiments, the engineered T cells are harvested 2 days after activation. In some embodiments, the engineered T cells are harvested 3 days after activation. In some embodiments, the engineered T cells are harvested 4 days after activation. In some embodiments, the engineered T cells are harvested 5 days after activation. In some embodiments, the engineered T cells are harvested 6 days after activation. In some embodiments, the engineered T cells are harvested 7 days after activation.

In some embodiments, the engineered T cells are harvested no later than 7 days after activation. In some embodiments, the engineered T cells are harvested no later than 6 days after activation. In some embodiments, the engineered T cells are harvested no later than 5 days after activation.

In some embodiments, the engineered T cell is harvested no later than 7 days after activation.

In some embodiments, the engineered T cell is harvested no later than 6 days after activation.

In some embodiments, the engineered T cell is harvested no later than 5 days after activation.

In some embodiments, the method further comprises transfecting or transducing the engineered T cell with a nucleic acid molecule encoding for a heterologous molecule of interest.

In some embodiments, the method further comprises transfecting the engineered T cell with a nucleic acid molecule encoding for a heterologous molecule of interest.

In some embodiments, the method further comprises transducing the engineered T cell with a nucleic acid molecule encoding for a heterologous molecule of interest.

In some embodiments, the heterologous molecule of interest is an siRNA, an shRNA, a non-coding RNA (e.g. a guide RNA for a CRISPR system), a peptide, a polypeptide, a protein, a viral payload, a viral genome, or a combination thereof.

In some embodiments, the heterologous molecule of interest is an siRNA. In some embodiments, the heterologous molecule of interest is an shRNA. In some embodiments, the heterologous molecule of interest is a non-coding RNA (e.g. a guide RNA for a CRISPR system). In some embodiments, the heterologous molecule of interest is a peptide. In some embodiments, the heterologous molecule of interest is a polypeptide. In some embodiments, the heterologous molecule of interest is a protein. In some embodiments, the heterologous molecule of interest is a viral payload. In some embodiments, the heterologous molecule of interest is a viral genome.

In some embodiments, the heterologous molecule of interest is a chimeric antigen receptor (CAR), also known as a chimeric immunoreceptor, chimeric T cell receptor, or artificial T cell receptor. In some embodiments, the heterologous molecule of interest is a CAR T cell.

In some embodiments, the heterologous molecule of interest is a chimeric antigen receptor (“CAR”). In some embodiments, the CAR T cells are CD19 CAR T cells. In some embodiments, the CAR T cells are CD22 CAR T cells. In some embodiments, the CAR T cells are CD5 CAR T cells. The terminology refers to the antigen to which the CAR binds. For example, a CD5 CAR T cell, refers to a T cell that comprises a CAR that comprises an antigen binding domain that binds to CD5.

In some embodiments, a synthetic control mechanism is added to the engineered T cells. In some embodiments, the control mechanism is the use of suicide genes. In some embodiments, the control mechanism is the use of dual-antigen receptors. In some embodiments, the control mechanism is an ON-switch. In some embodiments, the control mechanism is bispecific molecules as switches.

In some embodiments, the CAR T cells bear either of two types of co-receptors. In some embodiments, the CAR T cells bear CD4 and CD8. In some embodiments, the CAR T cells are CD4+. In some embodiments, the CAR T cells are CD8+.

In some embodiments, the CAR comprises an antigen recognition domain, an extracellular hinge region, a transmembrane domain, and an intracellular T cell signaling domain.

In some embodiments, the step of transfecting or transducing occurs after activation of the T cell.

In some embodiments, the T cell is transfected or transduced less than 1 day after activation of the T cell.

In some embodiments, the T cell is transfected or transduced more than 1 day after activation of the T cell.

In some embodiments, the T cell is transfected 1 day after activation of the T cell.

In some embodiments, the engineered T-cell is a patient-derived T cell.

In some embodiments, the engineered T-cell is derived autologously or allogeneically.

In some embodiments, the T cell is derived autologously from a subject to which the T cell will be administered to. In some embodiments, the T cell is derived allogeneically as compared to the subject to which the T cell is administered.

In some embodiments, the patient is a cancer patient.

In some embodiments, the cancer is a Blood cancer (hematologic malignancies) such as Leukemia (Acute Lymphoblastic Leukemia (ALL), Acute Myeloid Leukemia (AML), Chronic Lymphocytic Leukemia (CLL), Chronic Myeloid Leukemia (CML), Hairy Cell Leukemia, T-cell Prolymphocytic Leukemia (T-PLL), Large Granular Lymphocytic Leukemia (LGL)), Lymphoma (Hodgkin Lymphoma (HL), Non-Hodgkin Lymphoma (NHL), Diffuse Large B-Cell Lymphoma (DLBCL), Follicular Lymphoma (FL), Mantle Cell Lymphoma (MCL), Burkitt Lymphoma, Marginal Zone Lymphoma, Small Lymphocytic Lymphoma (SLL), T-Cell Lymphomas (including Peripheral T-cell Lymphoma, Anaplastic Large Cell Lymphoma)), Multiple Myeloma, Myeloproliferative Neoplasms (MPN) (Polycythemia Vera, Essential Thrombocythemia, Myelofibrosis), Myelodysplastic Syndromes (MDS), Solid tumors, such as Carcinomas (originating in epithelial cells) (Lung Cancer (Non-Small Cell Lung Cancer (NSCLC), Small Cell Lung Cancer (SCLC)), Breast Cancer, Colorectal Cancer, Prostate Cancer, Pancreatic Cancer, Gastric Cancer (Stomach Cancer), Liver Cancer (Hepatocellular Carcinoma), Esophageal Cancer, Ovarian Cancer, Endometrial Cancer (Uterine Cancer), Cervical Cancer, Head and Neck Cancer, Bladder Cancer, Kidney Cancer (Renal Cell Carcinoma), Thyroid Cancer, Adrenal Cancer), Sarcomas (arising in connective tissues) (Soft Tissue Sarcomas (e.g., Leiomyosarcoma, Liposarcoma, Angiosarcoma, Rhabdomyosarcoma), Bone Sarcomas (e.g., Osteosarcoma, Ewing's Sarcoma, Chondrosarcoma)), Brain Tumors (Gliomas (Astrocytoma, Glioblastoma Multiforme (GBM), Oligodendroglioma), Meningiomas, Ependymomas, Medulloblastomas, Pituitary Tumors), Skin Cancers (Melanoma, Basal Cell Carcinoma, Squamous Cell Carcinoma, Merkel Cell Carcinoma), Neuroendocrine Tumors (NETs) (Carcinoid Tumors, Pheochromocytoma, Paraganglioma, Pancreatic Neuroendocrine Tumors), Germ Cell Tumors (Testicular Cancer, Ovarian Germ Cell Tumors), Mesothelioma, Gastrointestinal Stromal Tumors (GIST), Thymoma and Thymic Carcinoma, Uveal Melanoma (Eye Cancer), Penile Cancer, Vaginal Cancer, Vulvar Cancer, Anal Cancer, Nasopharyngeal Carcinoma, Pleural and Peritoneal Cancer, Cholangiocarcinoma (Bile Duct Cancer), Gallbladder Cancer, Wilms Tumor (Nephroblastoma, Kidney Cancer in children), Retinoblastoma (Eye Cancer in children), Rhabdoid Tumor (Rare, aggressive pediatric cancer), Hepatoblastoma (Liver cancer in children), or Langerhans Cell Histiocytosis.

In some embodiments, the cancer patient is a lymphoma patient.

In some embodiments, the lymphoma patient T cells are harvested through leukocyte apheresis (i.e leukapheresis).

In some embodiments, the harvested lymphoma patient T cells undergo a 5-day manufacturing process.

In some embodiments, a manufacturing process is provided that comprises the steps of gene editing a population of T cells, activating the gene edited T cells, transducing the activated T cells, and optionally cryopreserving the transduced cells. In some embodiments, the process is completed within 5 days.

In some embodiments, manufacturing process comprises the steps in the following order: (i) gene editing a population of T cells; (ii) activating the gene edited T cells; (iii) transducing the activated T cells; and (iv) optionally cryopreserving the transduced cells.

In some embodiments, the patient T cells are enriched prior to undergoing gene editing.

In some embodiments, the enriched T cells are CD4+/CD8+ T cells.

In some embodiments, the T cells are enriched with CD4/CD8 microbeads.

In some embodiments, the cell is gene edited with a zinc finger nuclease system (ZFN), transcription activator-like effector nucleases (TALENs), CRISPR-Cas, base editing, prime editing, or programmable addition via site-specific targeting elements (PASTE).

In some embodiments, the cell is gene edited with a zinc finger nuclease system (ZFN). In some embodiments, the cell is gene edited with transcription activator-like effector nucleases (TALENs). In some embodiments the cell is gene edited with the CRISPR-Cas system. In some embodiments, the cell is gene edited through base editing. In some embodiments, the cell is gene edited through prime editing. In some embodiments, the cell is gene edited through programmable addition via site-specific targeting elements (PASTE).

In some embodiments, the gene editing system is the CRISPR-Cas system. In some embodiments, the CRISPR-Cas system is a Class 1 or Class 2 CRISPR-Cas system. In some embodiments, the CRISPR-Cas system is a Class 1 system. In some embodiments, the CRISPR-Cas system is a Class 2 system. In some embodiments the Class 2 system comprises a Type II Cas Protein. In some embodiments the Class 2 system comprises a Class V Cas Protein. In some embodiments the Class 2 system comprises a Class VI Cas Protein. In some embodiments, the Class 2 CRISPR-Cas system comprises a Type II Cas protein. In some embodiments, the Type II Cas protein is a Cas9 protein. In some embodiments, the CRISPR-Cas system utilizes a True-cut Cas9 nuclease. In some embodiments, the CRISPR-Cas system utilizes a high fidelity (HiFi) Cas 9. In some embodiments, the HiFi Cas9 utilizes a R691A HiFi Cas9 Variant. In some embodiments, the R691A HiFi Cas9 variant reduces off-target effects of gene editing. Other examples of nucleases that can be used, include, but are not limited to, an endonuclease comprising a RuvC domain or HEPN domain. In some embodiments, the endonuclease is a Cas12a endonuclease. In some embodiments, the endonuclease is not a Cas12a endonuclease. Non-limiting examples of nucleases are also provided for in WO2021178933, WO2021226363, WO2023039377. WO2023039378, WO/2023/028348, WO/2022/256462, WO/2022/256462, WO2022159742, WO/2022/159758, WO/2022/056301, WO/2022/056324, WO/2021/226363, WO/2021/226369, WO/2021/202568, WO/2021/202559, WO/2021/178933, WO/2021/178934, WO/2020/168291, or WO/2020/168234, each of which is hereby incorporated by reference in its entirety.

In some embodiments, the gene editing system comprises a targeting molecule that binds to a target sequence in an early exon or intron of the CD5 gene. In some embodiments, the gene editing system comprises a targeting molecule that binds to a target sequence in a late exon or intron of the CD5 locus. In some embodiments, the gene editing system comprises a targeting molecule that binds to a target sequence of the CD5 locus, and the target sequence is downstream of a preantepenultimate exon, e.g., is in an antepenultimate exon, a penultimate exon, or a last exon of the CD5 locus. Examples of which can be found in US2022/0073639, which is hereby incorporated by reference in its entirety.

In some embodiments, the gene editing system is selected from the group consisting of: a CRISPR or CRISPR/Cas9 system, a zinc finger nuclease system, a TALEN system, and a meganuclease system.

In some embodiments, the gene editing is performed through use of non-viral transfection. In some embodiments, the gene editing is performed by electroporation. In some embodiments, the electroporation is flow electroporation using a flow electroporation device. In the embodiments disclosed with respect to the flow electroporation device, it is specifically contemplated that the stated parameters and parameter ranges for flow electroporation are applicable to static electroporation devices used in the methods set forth herein. In specific embodiments, flow electroporation is used and static electroporation or non-flow electroporation is excluded. In a further specific embodiment, static electroporation is used and flow electroporation is excluded. For example guide RNAs can be mixed with the nuclease to form a ribonucleoprotein (RNP) complex prior to electroporation. The complex can then be electroporated into the cell to gene edit the cell. The guide RNA can target any target of interested to be edited. For example, the target can be CD5, CD2, or CD7. In some embodiments, the target is CD5. Examples of guide RNAs that can be used are described in US2022/0073639, which is hereby incorporated by reference in its entirety.

In some embodiments, the gene editing step occurs within 24 hours prior to activation of the unstimulated T cells. In some embodiments, the gene editing step occurs 24 hours prior to activation. In some embodiments, the gene editing step occurs 23 hours prior to activation. In some embodiments, the gene editing step occurs 22 hours prior to activation. In some embodiments, the gene editing step occurs 21 hours prior to activation. In some embodiments, the gene editing step occurs 20 hours prior to activation. In some embodiments, the gene editing step occurs 19 hours prior to activation. In some embodiments, the gene editing step occurs 18 hours prior to activation. In some embodiments, the gene editing step occurs 17 hours prior to activation. In some embodiments, the gene editing step occurs 16 hours prior to activation. In some embodiments, the gene editing step occurs 15 hours prior to activation. In some embodiments, the gene editing step occurs 14 hours prior to activation. In some embodiments, the gene editing step occurs 13 hours prior to activation. In some embodiments, the gene editing step occurs 12 hours prior to activation. In some embodiments, the gene editing step occurs 11 hours prior to activation. In some embodiments, the gene editing step occurs 10 hours prior to activation. In some embodiments, the gene editing step occurs 9 hours prior to activation. In some embodiments, the gene editing step occurs 8 hours prior to activation. In some embodiments, the gene editing step occurs 7 hours prior to activation. In some embodiments, the gene editing step occurs 6 hours prior to activation. In some embodiments, the gene editing step occurs 5 hours prior to activation. In some embodiments, the gene editing step occurs 4 hours prior to activation. In some embodiments, the gene editing step occurs 3 hours prior to activation. In some embodiments, the gene editing step occurs 2 hours prior to activation. In some embodiments, the gene editing step occurs 1 hour prior to activation.

As provided for herein, activation can occur through the use of any reagent sufficient to activate T cells or the cells of interest. For example, the cells can be activated with anti-CD3 and/or anti-CD28 antibodies. These can be coated onto microspheres, particles or beads, such as nanosized particles coated with anti-CD3 and anti-CD28 antibodies or be added without beads or particles (e.g., Enceed™ (Genscript); Dynabeads; ImmunoCult™). Other reagents that can be used either alone or in conjunction with anti-CD3 and anti-CD28 antibodies include concanavalin phorbol 12-myristate 13-acetate (PMA), ionomycin, brefeldin A, and monensin. In some embodiments, the cells are activated with a combination of antibodies directed CD2, CD3, and CD28. In some embodiments, these antibodies are biotinylated. In some embodiments, the cells are activated in the presence of anti-biotin beads that bind to the biotinylated antibodies (e.g., T Cell Activation/Expansion Kit, human; Miltenyl Biotec). Other reagents include, soluble anti-CD3/CD28 Fab fragments linked to a recombinant Streptactin backbone (e.g, Expamer™)

In some embodiments, the T cell population undergoes activation through antigen-induced activation. In some embodiments, the enriched T cell population undergoes non antigen-induced activation. In some embodiments, the enriched T cell population is activated through the use of CD3/CD28 antigen activation, CD3/CD28/CD137 antigen activation, Concanavalin A (conA), phorbol 12-myristate 13-acetate (PMA), or Treg expansion. In some embodiments, the enriched T cell population is activated through the use of CD3/CD28 antigen activation. In some embodiments, the enriched T cell population is activated through the use of CD3/CD28/CD137 antigen activation. In some embodiments, the enriched T cell population is activated through the use of Concanavalin A (conA). In some embodiments, the enriched T cell population is activated through the use of phorbol 12-myristate 13-acetate (PMA). In some embodiments, the enriched T cell population is activated through the use of Treg expansion. In some embodiments, the enriched T cell population undergoes antigen-induced activation. In some embodiments, the gene-edited patient T cells are activated through use of the «β-T-cell receptor (TCR) complex.

In some embodiments, the gene-edited, activated patient T cells undergo transduction, by, for example, a lentiviral vector. In some embodiments, the transduction is chimeric antigen receptor (CAR) transduction, which refers to a transduction of a heterologous nucleic acid molecule encoding the chimeric antigen receptor (CAR).

In some embodiments, the methods provided for herein produce a population of cells that is heterogenous. For example, the population of cells can be comprise a first population of cells that comprise a CAR and the gene edit and a second population of cells that comprises the gene edit but does not comprise the nucleic acid molecule encoding the CAR or the CAR itself.

The cell can be produced either ex vivo or in vivo. Methods for extracting, expanding and activating T cells are known in the art. The cells can be modified to express the heterologous sequence either ex vivo or in vivo. The cell can be contacted with plasmids or virus vectors comprising the components to be transduced or transfected into the cell. For example, a virus, such as a lentivirus (can be a plurality of viruses), can be used to transduce an immune cell either ex vivo or in vivo to edit the cell such that the heterologous nucleic acid molecule is inserted into the genome of the cell.

The present compositions and cells can be used to treat a subject with cancer, the method comprising administering to the subject a cell or composition as provided for herein.

In some embodiments, the CAR-mediated T-cell response can be directed to an antigen of interest by way of engineering an antigen binding domain that specifically binds a desired antigen into the CAR.

In some embodiments, the portion of the CAR comprising the antigen binding domain comprises an antigen binding domain that targets a tumor antigen, e.g., a tumor antigen described herein.

In some embodiments, the cell population comprises the CAR or the heterologous protein of interest. In some embodiments, the cell population does not comprise the CAR or the heterologous protein of interest.

In some embodiments, the methods further comprise isolating or harvesting cells that have been genetically modified, activated, and/or transduced with a heterologous molecule of interest. In some embodiments, the isolated cells are frozen or cryopreserved. In some embodiments, the cryopreservation occurs on day 5 after the gene editing step.

In some embodiments, the CAR-T cell is cryopreserved 24 hours after CAR transduction. In some embodiments, the CAR-T cell is cryopreserved 48 hours after CAR transduction. In some embodiments, the CAR-T cell is cryopreserved 72 hours after CAR transduction. In some embodiments, the CAR-T cell is cryopreserved 96 hours after CAR transduction. In some embodiments, the CAR-T cell is cryopreserved 120 hours after CAR transduction. In some embodiments, the CAR-T cell is cryopreserved 24-120 hours after CAR transduction. In some embodiments, the CAR-T cell is cryopreserved 24-132 hours after CAR transduction. In some embodiments, the CAR-T cell is cryopreserved 24-148 hours after CAR transduction.

In some embodiments, the gene-edited, activated, and/or transduced T cells are infused back into a patient. The cells can be autologous or allogeneic to the patient. As provided for herein, the patient can be a cancer patient or is suspected of having cancer. Non-limiting examples of cancers are provided for herein. In some embodiments, the cancer is lymphoma.

In some embodiments, a method of manufacturing a CAR T cell comprises the steps of harvesting patient T cells through leukapheresis in a T-cell lymphoma patient, electroporating the T cells with a gene editing complex to genetically modify the T-cells on day 0, activating the gene edited T-cells within 24 hours of electroporation by CD3/CD28 microbead antigen-induced activation, transducing the genetically modified, activated T cells within 24 hours of activation with a CAR, and, optionally, cryopreserving the CAR-T cell. In some embodiments, the methods comprise infusing the cells into a patient. In some embodiments, if the cells are frozen, the cells are thawed prior to infusing the cells into a patient.

### ENUMERATED EMBODIMENTS

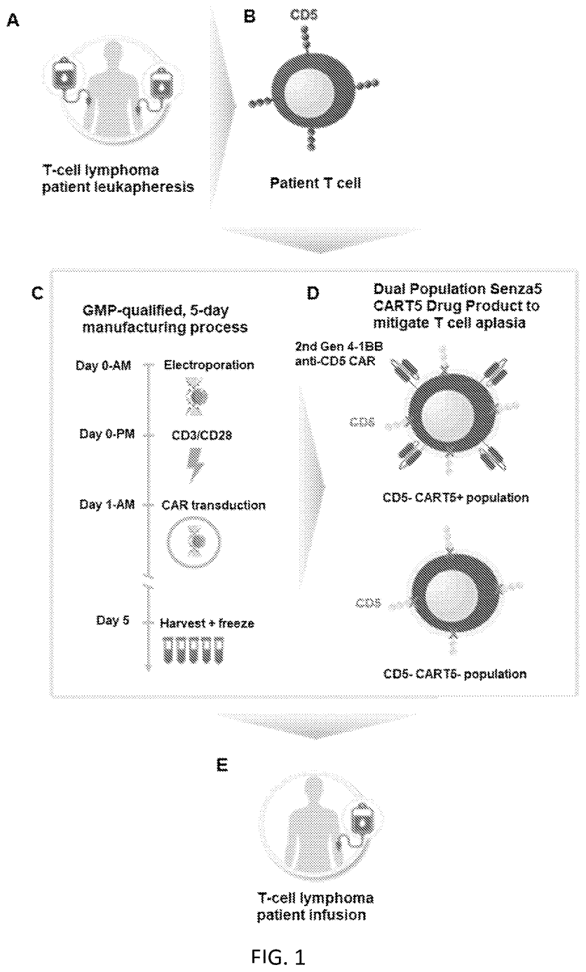

In some embodiments, the following embodiments are provided:
1. A method of preparing a T cell, the method comprising genetically editing an unstimulated T cell.
2. A method of preparing a T cell, the method comprising genetically editing an unstimulated T cell using an ex vivo manufacturing process wherein the T cells are in culture for up to seven days.
3. The method of embodiment 1 or 2, the method further comprising activating the genetically edited T cell.
4. The method of embodiment 3, wherein activating the genetically edited T cell comprises contacting the gene edited unstimulated T cell population with anti-CD3 and anti-CD28 antibodies and/or activating the genetically edited T cell comprises activating the cells through use of the αβ-T-cell receptor (TCR) complex.
5. The method of any one of embodiments 1-4, wherein the cell is genetically edited to modify a T cell genetic locus.
6. The method of embodiment 4, wherein the genetic locus is a CD5 genetic locus.
7. The method of embodiment 6, wherein the CD5 genetic locus is an exon, an intron, or a non-coding region of the CD5 genetic locus.
8. The method of any one of embodiments 1-7, wherein the genetically editing the T cell comprises contacting the T cell with a gene editing complex to genetically edit the T cell.
9. The method of embodiment 8, wherein the gene editing complex comprises a ribonucleoprotein complex.
10. The method of embodiments 8 or 9, wherein the gene editing complex comprises a nuclease and/or a guide RNA (gRNA).
11. The method of any one of embodiments 8-10, wherein the nuclease is a zinc finger nuclease system (ZFN), a transcription activator-like effector nuclease (TALEN), a CRISPR-Cas nuclease, a base editing nuclease, a prime editing nuclease, a retron based nuclease, or programmable addition via site-specific targeting element nuclease (PASTE).
12. The method of embodiment 11, wherein the CRISPR-Cas nuclease is a Class 1 or Class 2 CRISPR-Cas nuclease.
13. The method of embodiment 12, wherein the Class 2 CRISPR-Cas system comprises a Type II Cas nuclease.
14. The method of embodiment 13, wherein the Type II Cas nuclease is a Cas9 nuclease.
15. The method of any one of embodiments 10-13, wherein the CRISPR-Cas nuclease is a high fidelity (HiFi) nuclease, such as a high-fidelity Cas 9 nuclease.
16. The method of embodiment 15, wherein the HiFi Cas9 comprises a R691A substitution.
17. The method of embodiments 15 or 16, wherein the high fidelity nuclease reduces off-target gene editing.
18. The method of embodiment 10, wherein the nuclease is an endonuclease comprising a RuvC domain or HEPN domain, wherein said endonuclease is derived from an uncultivated microorganism, and wherein said endonuclease is:
   a Cas12a endonuclease;
   is not a Cas12a endonuclease,
   or wherein the nuclease is as provided for in WO2021178933, WO2021226363, WO2023039377. WO2023039378, WO/2023/028348, WO/2022/256462, WO/2022/256462, WO2022159742, WO/2022/159758, WO/2022/056301, WO/2022/056324, WO/2021/226363, WO/2021/226369, WO/2021/202568, WO/2021/202559, WO/2021/178933, WO/2021/178934, WO/2020/168291, or WO/2020/168234.
19. The method of any one of embodiments, 8-18, wherein the contacting comprises transfecting (e.g., non-viral transfection) or electroporating the cell with the gene editing complex.
20. The method of embodiment 19, wherein the gene editing complex comprises a lipoparticle (e.g., liponanoparticle) encapsulated gene editing complex, or a portion thereof.
21. The method of embodiments 19 or 20, wherein the contacting comprises electroporating the cell.
22. The method of embodiment 21, wherein the electroporating comprises electroporating the cell with a flow electroporation device or system.
23. The method of embodiment 22, wherein the electroporation device or system is as described in WO2005113820, WO2003018751, WO2003018751, WO2010009252, WO2021231497, WO2006060409, WO2020086701, WO2022232802, WO/2004/031353, WO/2007/021993, or WO/2007/021994.
24. The method of embodiments 1-23, wherein the gene editing comprises contacting the cell with a gene editing complex for about 30 minutes to about 72 hours before activating the gene edited cell, about 6 hours to about 36 hours before activating the gene edited cell, about 12 hours to about 36 hours before activating the gene edited cell, about 18 hours to about 28 hours before activating the gene edited cell, about 20 hours to about 26 hours before activating the gene edited cell, or about 24 hours before activating the gene edited cell.
25. The method of any one of embodiments 1-23, wherein the gene editing comprises contacting the cell with a gene editing complex about 30 minutes to about 72 hours before activating the gene edited cell.
26. The method of embodiment 25, wherein the gene editing wherein the gene editing comprises contacting the cell with a gene editing complex about 6 hours to about 36 hours before activating the gene edited cell.
27. The method of embodiment 25, wherein the gene editing wherein the gene editing comprises contacting the cell with a gene editing complex about 12 hours to about 36 hours before activating the gene edited cell.
28. The method of embodiment 25, wherein the gene editing wherein the gene editing comprises contacting the cell with a gene editing complex about 18 hours to about 28 hours before activating the gene edited cell.
29. The method of embodiment 25, wherein the gene editing wherein the gene editing comprises contacting the cell with a gene editing complex about 20 hours to about 26 hours before activating the gene edited cell.
30. The method of embodiment 25, wherein the gene editing wherein the gene editing comprises contacting the cell with a gene editing complex about 24 hours before activating the gene edited cell.
31. The method of any one of embodiments 1-30, the method further comprising contacting the gene-edited activated T cell with a vector comprising a heterologous nucleic acid molecule encoding a molecule of interest.
32. The method of embodiment 31, wherein the vector is a plasmid or a viral vector.
33. The method of embodiment 32, wherein the viral vector is lentivirus based viral vector.
34. The method of any one of embodiment 1-33, wherein the method further comprises harvesting the activated T cell.
35. The method of any one of embodiments 1-34, wherein the activated T cell is harvested no later than 7 days after activation.
36. The method of any one of embodiments 1-34, wherein the activated T cell is harvested between 1 and 7 days after activation.
37. The method of any one of embodiments 1-34, wherein the activated T cells is harvested 1, 2, 3, 4, 5, 6, or 7 days after activation.
38. The method of any one of embodiments 1-34, wherein the activated T cell is harvested no later than 6 days after activation.
39. The method of any one of embodiments 1-34, wherein the activated T cell is harvested no later than 5 days after activation.
40. The method of any one of embodiments 31-33, wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.
41. The method of any one of embodiments 31-33, wherein the heterologous molecule of interest is a chimeric antigen receptor (“CAR”).
42. The method of embodiment 41, wherein the CAR comprises an antigen binding domain that binds to CD5.
43. The method of embodiment 42, wherein the antigen binding domain against CD5 comprises a heavy chain variable region (VH) and a light chain variable region (VL), wherein the VH comprises a heavy chain complementarity determining region 1 (HCDR1) comprising the amino acid sequence of SEQ ID NO: 1, a HCDR2 comprising the amino acid sequence of SEQ ID NO: 2, a HCDR3 comprising the amino acid sequence of SEQ ID NO: 3; and wherein the VL comprises a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6.
44. The method of embodiment 43, wherein the VH comprises an amino acid sequence having at least 80%, 85%, 90%, 95%, 98%, 99%, or 100% identity to the amino acid sequence of SEQ ID NO: 7; and the VL comprises an amino acid sequence having at least 80%, 85%, 90%, 95%, 98%, 99%, or 100% identity to the amino acid sequence of SEQ ID NO: 8.
45. The method of embodiment 42, wherein the CAR comprises an amino acid sequence having at least 80%, 85%, 90%, 95%, 98%, 99%, or 100% identity to the amino acid sequence of SEQ ID NO: 11.
46. The method of embodiment 42, wherein the CAR comprises an amino acid sequence having at least 80%, 85%, 90%, 95%, 98%, 99%, or 100% identity to the amino acid sequence of SEQ ID NO: 12.
47. The method of any one of embodiments 31-46, wherein the T cell is transfected or transduced less than 1 day after activation of the T cell.
48. The method of any one of embodiments 31-46, wherein the T cell is transfected or transduced more than 1 day after activation of the T cell.
49. The method of any one of embodiments 1-48, wherein the engineered T-cell is a patient-derived T cell.
50. The method of embodiment 49, wherein the patient is a cancer patient.
51. The method of embodiment 50, wherein the cancer is a blood cancer, or hematologic malignancy, including leukemia (acute lymphoblastic leukemia (ALL), acute myeloid leukemia (AML), chronic lymphocytic leukemia (CLL), and chronic myeloid leukemia (CML)), lymphoma (Hodgkin lymphoma (HL) and non-Hodgkin lymphoma (NHL) with subtypes like diffuse large B-cell lymphoma (DLBCL), follicular lymphoma (FL), and mantle cell lymphoma (MCL)), and multiple myeloma, or a solid tumor comprising carcinomas (originating in epithelial cells), sarcomas (arising in connective tissues), and various other types such as brain tumors (gliomas, meningiomas), breast cancer, lung cancer, colorectal cancer, prostate cancer, pancreatic cancer, melanoma (skin cancer), kidney cancer, bladder cancer, ovarian cancer, or endometrial cancer, among others.
52. The method of embodiments 50 or 51, wherein the cancer patient is a lymphoma patient.
53. The method of any one of embodiments 1-52, wherein the method comprises isolating T cells from a patient, such as through leukapheresis, prior to gene editing the cells.
54. The method of any one of embodiments 1-53, wherein the method further cryopreserving the cells after any of the steps.
55. The method of any one of embodiments 1-54, wherein the T cells are enriched prior to undergoing gene editing.
56. The method of embodiment 55, wherein the enriched T cells are CD4+/CD8+ T cells, by, for example, the use of CD4/CD8 microbeads.
57. The method of any one of embodiments 1-56, wherein the T cell is a CD5+ T cell.
58. The method of embodiment 49-57, wherein the T cell population is activated through the use of CD3/CD28 antigen activation, CD3/CD28/CD137 antigen activation, Concanavalin A (conA), phorbol 12-myristate 13-acetate (PMA), or Treg expansion.
59. The method of any one of embodiments 1-58, wherein the gene-edited activated T cells are administered to a patient.
60. A method of manufacturing a CAR-T cell comprising the steps of:
   gene editing isolated unstimulated T cells;
   electroporating the T cells with a gene editing complex to genetically edit the T cell to produce gene edited T cells;
   activating the gene edited T-cells within 24 hours of electroporation by CD3/CD28 microbead antigen-induced activation; and
   transducing the genetically modified, activated T cells within 24 hours of activation with a lentivirus based vector comprising a heterologous nucleic acid molecule encoding a chimeric antigen receptor.
61. The method of embodiment 60, wherein the method further comprises culturing the transduced T cells for a period of time, such as 1-5 days.
62. The method of embodiments 60 or 61, wherein the method further comprises freezing (cryopreserving) the transduced T cells.
63. The method of any one of embodiments 60-62, wherein the gene editing edits the CD5 locus to disrupt the CD5 locus, knock-out the CD5 gene, and/or reduce the expression of CD5.
64. A method of manufacturing a CAR-T cell comprising:
   i) isolating unstimulated T cells from a subject;
   ii) electroporating the unstimulated T cells with a gene editing complex to genetically edit the T cell to produce unstimulated, gene edited T cells;
   iii) activating the unstimulated, gene edited T-cells within 24 hours of electroporation by CD3/CD28 microbead antigen-induced activation to produce activated, gene edited T cells;
   iv) transducing the activated, gene edited T cells within 24 hours of activation with a lentivirus based vector comprising a heterologous nucleic acid molecule encoding a chimeric antigen receptor (“CAR”);
   v) culturing the T cells for 1 to 7 days after transductions;
   vi) harvesting the T cells 1 to 7 days after transduction; and
   vii) optionally, freezing the harvested cells.
65. The method of embodiment 64, wherein the CAR comprises an antigen binding domain that binds to CD5.
66. The method of embodiment 65, wherein the antigen binding domain against CD5 comprises a heavy chain variable region (VH) and a light chain variable region (VL), wherein the VH comprises a heavy chain complementarity determining region 1 (HCDR1) comprising the amino acid sequence of SEQ ID NO: 1, a HCDR2 comprising the amino acid sequence of SEQ ID NO: 2, a HCDR3 comprising the amino acid sequence of SEQ ID NO: 3; and wherein the VL comprises a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6.
67. The method of embodiment 66, wherein the VH comprises an amino acid sequence having at least 80%, 85%, 90%, 95%, 98%, 99%, or 100% identity to the amino acid sequence of SEQ ID NO: 7; and the VL comprises an amino acid sequence having at least 80%, 85%, 90%, 95%, 98%, 99%, or 100% identity to the amino acid sequence of SEQ ID NO: 8.
68. The method of embodiment 64, wherein the CAR comprises an amino acid sequence having at least 80%, 85%, 90%, 95%, 98%, 99%, or 100% identity to the amino acid sequence of SEQ ID NO: 11.
69. The method of embodiment 64, wherein the CAR comprises an amino acid sequence having at least 80%, 85%, 90%, 95%, 98%, 99%, or 100% identity to the amino acid sequence of SEQ ID NO: 12.
70. A CAR-T cell produced according to the method of any one of claims 1-69.
71. A method of treating a cancer patient with a CAR-T cell, the method comprising:
   gene editing isolated unstimulated T cells obtained from a subject;
   electroporating the T cells with a gene editing complex to genetically edit the T cell to produce gene edited T cells;
   activating the gene edited T-cells within 24 hours of electroporation by CD3/CD28 microbead antigen-induced activation;
   transducing the genetically modified, activated T cells within 24 hours of activation with a lentivirus based vector comprising a heterologous nucleic acid molecule encoding a chimeric antigen receptor; and
   administering the transduced T cells to the patient.
72. A method of treating a cancer patient with a CAR-T cell, the method comprising:
   i) isolating unstimulated T cells from a subject;
   ii) electroporating the unstimulated T cells with a gene editing complex to genetically edit the T cell to produce unstimulated, gene edited T cells;
   iii) activating the unstimulated, gene edited T-cells within 24 hours of electroporation by CD3/CD28 microbead antigen-induced activation to produce activated, gene edited T cells;
   iv) transducing the activated, gene edited T cells within 24 hours of activation with a lentivirus based vector comprising a heterologous nucleic acid molecule encoding a chimeric antigen receptor;
   v) culturing the T cells for 1 to 7 days after transductions;
   vi) harvesting the T cells 1 to 7 days after transduction;
   vii) optionally, freezing the harvested cells; and
   viii) administering the harvested T cells to the patient.
73. The method of embodiment 71 or 72, wherein the subject is the cancer patient or the subject is a person who is not the patient (e.g., the cells are allogeneic to the patient).
74. The method of embodiment 71 or 72, wherein the transduced cells are frozen prior to being administered to the patient.
75. The method of embodiment 74, wherein the frozen cells are thawed prior to being administered to the patient.
76. The method of any one of embodiments 71-75, wherein the method is performed according to the steps illustrated in FIG. 1.

The following examples are illustrative, but not limiting, of the compounds, compositions and methods described herein. Other suitable modifications and adaptations known to those skilled in the art are within the scope of the following embodiments.

### EXAMPLES

### Example 1: Gene Editing Prior to Activation in a Shortened CAR-T Manufacturing Process

T-cell lymphoma patient T cells are harvested through leukapheresis (FIG. 1A). The patient T-cell expressing CD5 then undergoes a manufacturing process, in which the cells are electroporated for gene editing to knock-out or reduce the expression of CD5 (FIG. 1C). The gene edited T cells are activated using CD3/CD28 microbeads, the cells undergo viral transduction to express the a chimeric antigen receptor comprising a CD5 antigen binding domain on the T cell. The transduced T cells are then harvested and frozen for long-term storage and/or clinical use. The manufacturing process results in a dual population of cells, what can be collectively referred to as the CART5 drug product. Within the two populations, there consists of cells that are CD5+CART5+ or CD5−CART5− (FIG. 1D). This drug product may then be infused back into the T-cell lymphoma patient to treat the lymphoma (FIG. 1E). This method is illustrated, for example, in FIG. 1.

### Example 2

Human T cells were procured and enriched for CD4+ and CD8+ positive T cells (scale 1M to 1B). CD4+ and CD8+ T cells were combined at a 1:1 ratio and used for electroporation. CRISPR-Cas9 sgRNAs against CD5 were generated either through in vitro transcription using the GeneArt Precision gRNA Synthesis Kit (Invitrogen; Cat #A29377) or were chemically synthesized (Integrated DNA Technologies or Synthego). 5 μg sgRNA were premixed with 10 μg of TrueCut Cas9 Protein v2 (Invitrogen; Cat #A36499) or HiFi Cas9 for 10 minutes at room temperature to form a ribonucleoprotein (RNP) complex prior to electroporation. 10×10⁶ T cells in 100 μL of the buffer provided with P3 Primary Cell 4D-Nucleofector X Kit L (Lonza; Cat #V4XP-3024, or Maxcyte) were mixed with the RNP complex and subsequently electroporated using the pulse code EO-115 in a 4D-Nucleofector (Lonza; Cat #AAF-1002B, or Maxcyte). Mock KO cells were electroporated using the same procedure as described without the presence of an RNP complex. After electroporation, T cells were incubated at 37 °C for 24 hours and subsequently activated using CD3/CD28 Dynabeads (Gibco; Cat #40203D) or Transact at a ratio of 3 beads/cell. The following day, CAR lentiviral vectors were added to stimulated cultures at a multiplicity of infection between 1 and 3. Beads were removed between days 6-8 of stimulation, and cells were counted every other day using a Multisizer 3 Coulter Counter (Beckman) or Moxi GO II (Orflo) until growth kinetics and cell size demonstrated they had rested from stimulation. All T cells were initially grown with 20 ng/ml of supplemental cytokines IL-7 and IL-15 that was decreased to 0 ng/ml by the end of the expansion. The unstimulated cells were found to be effectively edited and transduced and that the gene editing efficiency and time to produce the transduced cells was significantly shortened as compared to gene editing stimulated T cells. This could not have been predicted or expected because prior experiments suggested that gene editing unstimulated T cells was not effective or sufficiently efficient. The methods provided for herein can be used to shorten the manufacturing CAR-T cells, such as to no more than 5 days, which can be lead to cost savings and other efficiencies.

### Example 3: In Vitro Assessment of Rapid Manufactured CAR-T Cells

CAR-T cells produced via the methods of Examples 1 and 2 were compared to traditionally developed CAR-T cells to determine if the different manufacturing protocols resulted in changes in CAR-T cell properties and/or function. Briefly, cells generated via the rapid protocol were electoporated with the RNP complex described in Example 2 above to knock out CD5 expression on protocol Day 0 (AM). T cells were activated with CD3/CD28 Dynabeads on protocol Day 0 (PM). Activated T cells were transduced with CAR lentiviral vectors to express a CD5 CAR on protocol Day 1 (AM).

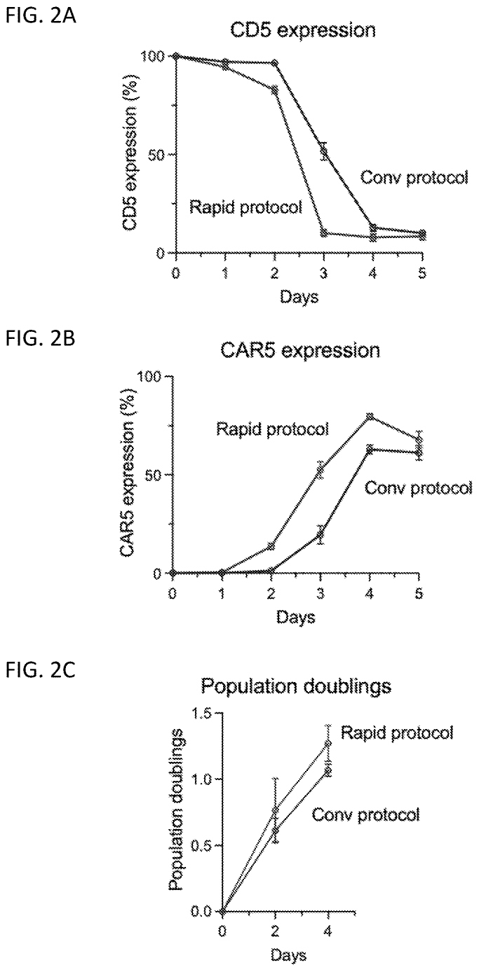

Beginning at protocol Day 0, cells undergoing the rapid and conventional protocols were assessed for CD5 expression (FIG. 2A), CD5 CAR (CAR5) expression (FIG. 2B), and for cell doubling rate. As illustrated in FIG. 2A, cells undergoing the rapid protocol demonstrated a more rapid decline in CD5 expression, consistent with the protocol design where T cells are receiving CD5 knock out machinery earlier in the process. Importantly, the rapid protocol and conventional protocol result in near equal CD5 knockdown, indicating that the rapid protocol is just as efficient as the conventional process. As illustrated in FIG. 2B, CAR5 expression also follows a similar trend, where the cells undergoing rapid protocol demonstrate a more rapid increase in CAR5 expression as compared to the conventional protocol. Here, the rapid protocol appears to result in a greater maximum percentage of CAR5 expressing cells, although both protocols show similar levels of CAR5 expression by protocol Day 5. These results demonstrate that the rapid protocol is just as effective at generating CAR5 positive cells as the conventional protocol. As illustrated in FIG. 2C, cells generated via rapid or conventional protocols exhibit similar doubling times, with the rapid protocol cells trending toward a quicker doubling time as compared to the conventional protocol. Overall, the results of FIG. 2A-C illustrate that the rapid manufacturing protocol results in equal knockdown of CD5 and expression of CAR5 as compared to the conventional protocol, indicating that the more rapid timescale does not negatively affect CAR5 cell production.

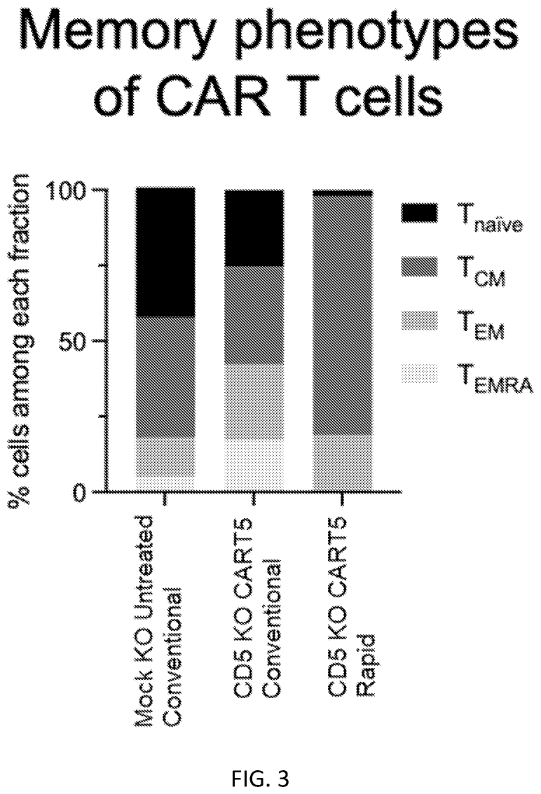

To assess whether the change in manufacturing protocol affected T cell properties, the memory phenotype and cytokine release profile of the T cells were assessed. Manufactured T cells were thawed from storage and the memory phenotype was assessed (FIG. 3). Naïve T cells are characterized as CD45RA⁺CCR7⁺. Central memory T cells are characterized as CD45RA⁻CCR7⁺. Effector memory T cells are characterized as CD45RA⁻CCR7⁻. Effector memory T cells re-expressing CD45RA are characterized as CD45RA⁺CCR7⁻. As illustrated in FIG. 3, mock knock out untreated T cells (control) generated via the conventional method comprised approximately 40% naïve T cells, 40% central memory T cells, 15% effector memory T cells, and 5% effector memory T cells re-expressing CD45RA. CD5 KO CART5 positive cells generated via conventional methods comprised approximately 25% naïve T cells, 35% central memory T cells, 25% effector memory T cells, and 15% effector memory T cells re-expressing CD45RA. CD5 KO CART5 positive cells generated via the rapid methods described herein comprised approximately 2% naïve T cells, 80% central memory T cells, 18% effector memory T cells, and <1% effector memory T cells re-expressing CD45RA. The results of these experiments illustrate that cells generated via the rapid method have a greatly reduced population of naïve T cells while also boasting a greatly increased population of memory T cells. Accordingly, these results suggest that the cells generated via the rapid method may result in a more robust effect when introduced to subjects due to the increased population of self-renewing T cells (i.e. memory cells).

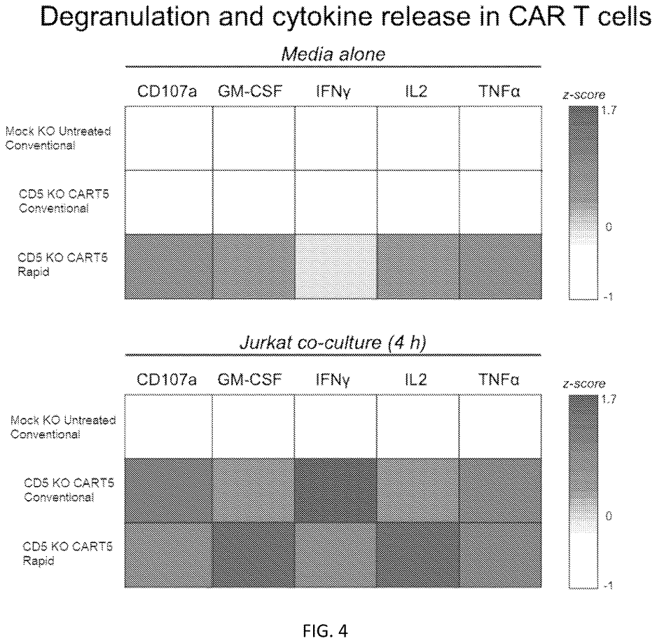

The cytokine release profile of the T cells was also assessed (FIG. 4). As illustrated in FIG. 4, mock knock out untreated T cells (control) generated via the conventional method and CD5 KO CART5 positive cells generated via the conventional method exhibit baseline levels of CD107a, GM-CSF, IFN gamma, IL-2 and TNF-alpha in media alone. After co-culture with Jurkat cells for 4 hours (E:T=1:1), mock cells showed no change in cytokine profile, while the CD5 KO CART5 cells demonstrated an increase in all cytokines assessed. In contrast, CD5 KO CART5 positive cells generated via the rapid method showed a higher than baseline level of each cytokine assessed when in media alone. Each cytokine assessed also exhibited an increase after co-culture with Jurkat cells for 4 hours (E:T=1:1). Interestingly, the relative cytokine levels between cytokines within a cell population was not the same between CD5 KO CART5 cells generated via conventional versus rapid methods. This could be due to the population of T cells making up the CD5 KO CART5 cell population, as discussed above and shown in FIG. 3.

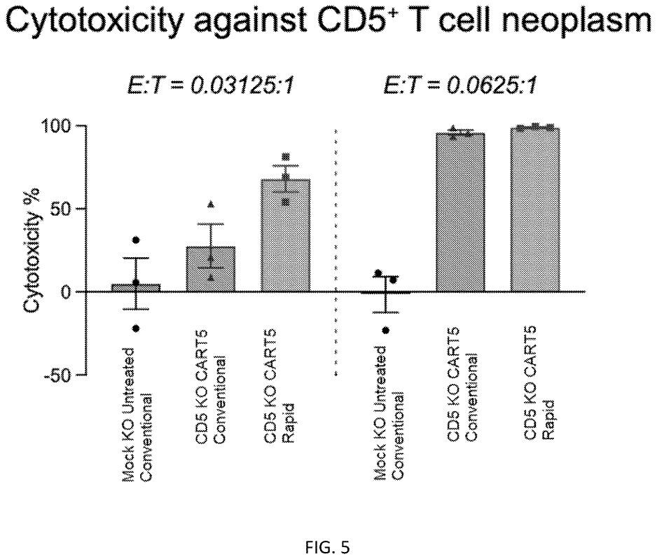

Having assessed the phenotypic characteristics of the CART5 cells generated via conventional and rapid methods, the functional differences between cells generated via the two methods was next assessed. First, the cytotoxicity of the CART5 cells against CD5+ Jurkat cells in co-culture was assessed. Mock cells, CD5 KO CART5 cells generated via conventional methods, and CD5 KO CART5 cells generated via the rapid method were incubated with CD5+ Jurkat cells for 72 hours at an E:T of either 0.03125:1 or 0.0625:1. As illustrated in FIG. 5, the CD5 KO CART5 cells generated via the rapid method were more potent against the CD5+ Jurkat cells at the lower ET ratio as compared to CD5 KO CART5 cells generated via conventional methods. Both cell populations were near equally potent at the higher E:T ratio. The increased potency of the CD5 KO CART5 cells generated via the rapid method at the lowed E:T ratio may be due to the difference in memory phenotype between the cell populations, as discussed above.

### Example 4: In Vitro Assessment of Rapid Manufactured CAR-T Cells

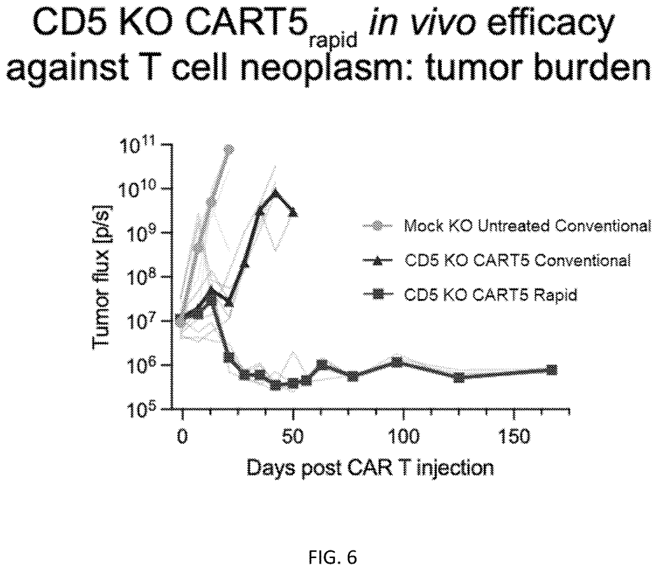

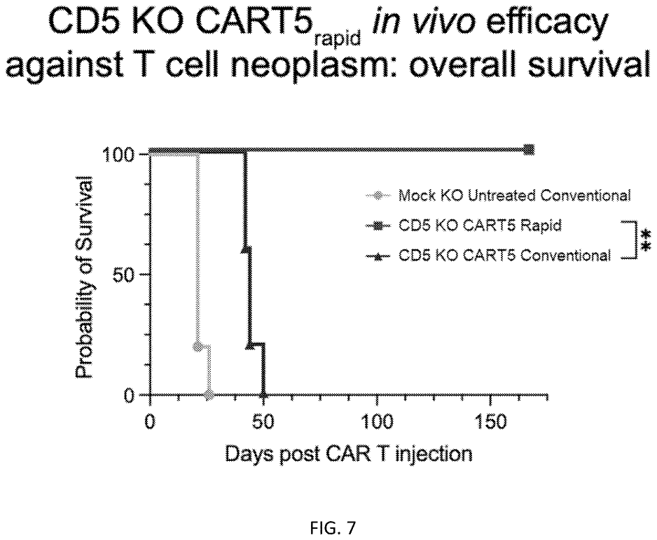

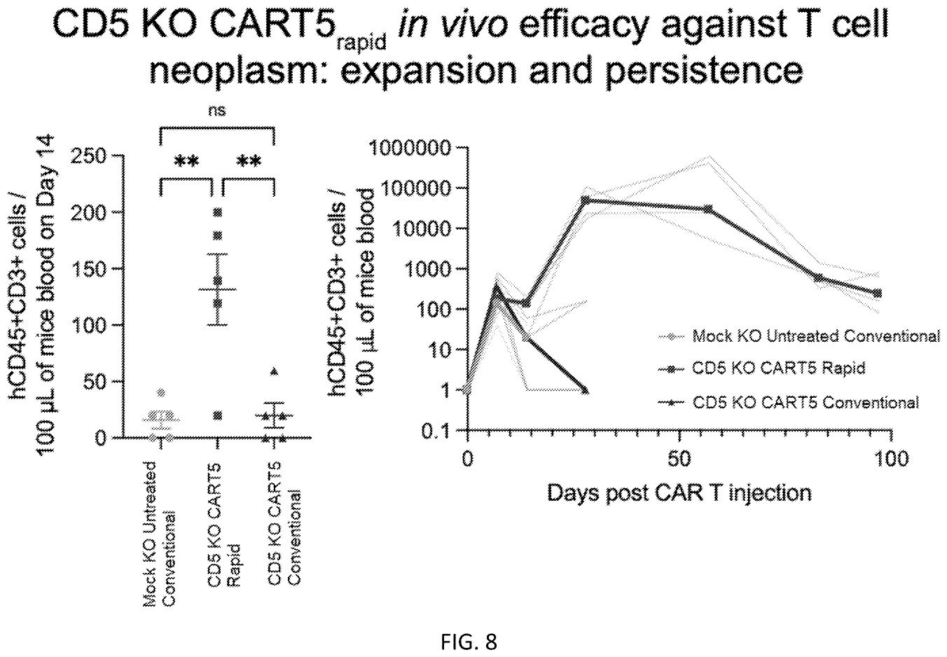

CAR-T cells produced via the methods of Examples 1 and 2 were compared to traditionally developed CAR-T cells in an NSG mouse tumor model. Briefly, NSG mice were engrafted with 1×10⁶ Jurkat cells (I.V.) on protocol day-7. Mice were then injected with 0.5×10⁶ Mock KO Untreated T cells, CD5 KO CART5 cells generated via conventional methods, or CD5 KO CART5 cells generated via the rapid method (I.V.) on protocol day 0, n=5 mice per group. Mice were then monitored for tumor burden (FIG. 6), overall survival (FIG. 7), and expansion and persistence of the CD5 KO CART5 cells (FIG. 8).

Tumor burden was assessed via bioluminescence imaging of the tumor in each NSG mouse. Results are illustrated in FIG. 6, where the bolded like represents the median of each group. As expected, mock KO untreated cells had no effect on tumor growth and progression. The CD5 KO CART5 cells generated via conventional methods slowed tumor growth for approximately 25 days post CAR T injection, whereafter the tumor burden increased. Surprisingly and unpredictably, the CD5 KO CART5 cells generated via the rapid method resulted in a reduction in tumor burden that was sustained for the duration of the study.

Animal survivability results reflected the tumor burden results discussed above. As illustrated in FIG. 7, mice receiving mock KO untreated cells had a rapid decline in probability of survival. Mice receiving CD5 KO CART5 cells generated via conventional methods displayed delayed mortality as compared to the mock KO animals. Surprisingly and unpredictably, none of the mice receiving CD5 KO CART5 cells generated via the rapid method succumbed to the tumor burden in this study.

The expansion and persistence of CAR T cells was assessed via absolute cell counts of hCD45+hCD3+ T cells in 100 μL of blood at set intervals (FIG. 8). As illustrated in the left panel of FIG. 8, at 14 days post CAR T cell injection there was an insignificant difference in hCD45+hCD3+ cell count in the blood of animals receiving mock KO cells and CD5 KO CART5 cells generated via conventional methods. However, in animals receiving CD5 KO CART5 cells generated via rapid methods there was a significant increase in hCD45+hCD3+ cell count as compared to the other two groups. This elevated cell count was maintained throughout the duration of the study (FIG. 8, right panel).

The results of the in vivo assessment of CD5 KO CART5 cells generated via conventional or rapid methods illustrates the surprising and unpredictable superior performance of the CD5 KO CART5 cells generated via rapid methods. The rapidly generated cells exhibited a decrease in tumor burden, a 100% survival rate for the duration of the study, and sustained CART5 expression. These results suggest that CD5 CART cells generated via the rapid method are superior to conventionally generated CAR T cells.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed:

1. A method of preparing a T cell, the method comprising genetically editing an unstimulated T cell.

2. A method of preparing a T cell, the method comprising genetically editing an unstimulated T cell using an ex vivo manufacturing process wherein the T cells are in culture for up to seven days.

3. The method of claim 1, the method further comprising activating the genetically edited T cell.

4. The method of claim 1, wherein activating the genetically edited T cell comprises contacting the gene edited unstimulated T cell population with anti-CD3 and anti-CD28 antibodies and/or activating the genetically edited T cell comprises activating the cells through use of the αβ-T-cell receptor (TCR) complex.

5. The method of claim 1, wherein the cell is genetically edited to modify a T cell genetic locus.

6. The method of claim 5, wherein the genetic locus is a CD5 genetic locus.

7. The method of claim 1, wherein the genetically editing the T cell comprises contacting the T cell with a gene editing complex to genetically edit the T cell.

8. The method of claim 7, wherein the gene editing complex comprises a ribonucleoprotein complex.

9. The method of claim 7, wherein the gene editing complex comprises a nuclease and/or a guide RNA (gRNA).

10. The method of claim 9, wherein the nuclease is a zinc finger nuclease system (ZFN), a transcription activator-like effector nuclease (TALEN), a CRISPR-Cas nuclease, a base editing nuclease, a prime editing nuclease, a retron based nuclease, or programmable addition via site-specific targeting element nuclease (PASTE).

11. The method of claim 10, wherein the CRISPR-Cas nuclease is a Class 1 or Class 2 CRISPR-Cas nuclease.

12. The method of claim 11, wherein the Class 2 CRISPR-Cas system comprises a Type II Cas nuclease.

13. The method of claim 12, wherein the Type II Cas nuclease is a Cas9 nuclease.

14. The method of claim 10, wherein the CRISPR-Cas nuclease is a high fidelity (HiFi) nuclease.

15. The method of claim 7, wherein the contacting comprises transfecting or electroporating the cell with the gene editing complex.

16. The method of claim 15, wherein the electroporating comprises electroporating the cell with a flow electroporation device or system.

17. The method of claim 1, wherein the gene editing comprises contacting the cell with a gene editing complex for about 30 minutes to about 72 hours before activating the gene edited cell, about 6 hours to about 36 hours before activating the gene edited cell, about 12 hours to about 36 hours before activating the gene edited cell, about 18 hours to about 28 hours before activating the gene edited cell, about 20 hours to about 26 hours before activating the gene edited cell, or about 24 hours before activating the gene edited cell.\

18. The method of claim 1, the method further comprising contacting the gene-edited activated T cell with a vector comprising a heterologous nucleic acid molecule encoding a molecule of interest.

19. The method of claim 18, wherein the vector is a plasmid or a viral vector.

20. The method of claim 19, wherein the viral vector is lentivirus based viral vector.

21. The method of claim 1, wherein the method further comprises harvesting the activated T cell.

22. The method of claim 21, wherein the activated T cell is harvested no later than 5 days after activation, no later than 6 days after activation, or no later than 7 days after activation.

23. The method of claim 21, wherein the activated T cell is harvested between 1 and 7 days after activation.

24. The method of claim 21, wherein the activated T cells is harvested 1, 2, 3, 4, 5, 6, or 7 days after activation.

25. The method of claim 18, wherein the heterologous molecule of interest is an siRNA, an shRNA, a non-coding RNA, a peptide, a polypeptide, a protein, a viral payload, a viral genome, or a combination thereof.

26. The method of claim 18, wherein the heterologous molecule of interest is a chimeric antigen receptor (“CAR”).

27. The method of claim 26, wherein the CAR comprises an antigen binding domain that binds to CD5.

28. The method of claim 27, wherein the antigen binding domain against CD5 comprises a heavy chain variable region (VH) and a light chain variable region (VL), wherein the VH comprises a heavy chain complementarity determining region 1 (HCDR1) comprising the amino acid sequence of SEQ ID NO: 1, a HCDR2 comprising the amino acid sequence of SEQ ID NO: 2, a HCDR3 comprising the amino acid sequence of SEQ ID NO: 3; and wherein the VL comprises a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6.

29. The method of claim 28, wherein the VH comprises an amino acid sequence having at least 80%, 85%, 90%, 95%, 98%, 99%, or 100% identity to the amino acid sequence of SEQ ID NO: 7; and the VL comprises an amino acid sequence having at least 80%, 85%, 90%, 95%, 98%, 99%, or 100% identity to the amino acid sequence of SEQ ID NO: 8.

30. The method of claim 26, wherein the CAR comprises an amino acid sequence having at least 80%, 85%, 90%, 95%, 98%, 99%, or 100% identity to the amino acid sequence of SEQ ID NO: 11.

31. The method of claim 26, wherein the CAR comprises an amino acid sequence having at least 80%, 85%, 90%, 95%, 98%, 99%, or 100% identity to the amino acid sequence of SEQ ID NO: 12.

32. The method of any one of claims 18-31, wherein the T cell is transfected or transduced less than 1 day after activation of the T cell.

33. The method of any one of claims 18-31, wherein the T cell is transfected or transduced more than 1 day after activation of the T cell.

34. The method of any one of claims 1-33, wherein the engineered T-cell is a patient-derived T cell.

35. The method of claim 34, wherein the patient is a cancer patient.

36. The method of claim 35, wherein the cancer is a blood cancer, or hematologic malignancy, including leukemia (acute lymphoblastic leukemia (ALL), acute myeloid leukemia (AML), chronic lymphocytic leukemia (CLL), and chronic myeloid leukemia (CML)), lymphoma (Hodgkin lymphoma (HL) and non-Hodgkin lymphoma (NHL) with subtypes like diffuse large B-cell lymphoma (DLBCL), follicular lymphoma (FL), and mantle cell lymphoma (MCL)), and multiple myeloma, or a solid tumor comprising carcinomas (originating in epithelial cells), sarcomas (arising in connective tissues), and various other types such as brain tumors (gliomas, meningiomas), breast cancer, lung cancer, colorectal cancer, prostate cancer, pancreatic cancer, melanoma (skin cancer), kidney cancer, bladder cancer, ovarian cancer, or endometrial cancer.

37. The method of claim 35 or 36, wherein the cancer patient is a lymphoma patient.

38. The method of any one of claims 1-37, wherein the method further comprises isolating T cells from a patient prior to gene editing the cells.

39. The method of any one of claims 1-38, wherein the method further comprises cryopreserving the cells after any of the steps.

40. The method of any one of claims 1-39, wherein the T cells are enriched prior to undergoing gene editing.

41. The method of any one of claims 1-40, wherein the T cell is a CD5+ T cell.

42. The method of claim 34, wherein the T cell population is activated through the use of CD3/CD28 antigen activation, CD3/CD28/CD137 antigen activation, Concanavalin A (conA), phorbol 12-myristate 13-acetate (PMA), or Treg expansion.

43. The method of any one of claims 1-42, wherein the gene-edited activated T cells are administered to a patient.

44. A method of manufacturing a CAR-T cell comprising the steps of:
gene editing isolated unstimulated T cells;
electroporating the T cells with a gene editing complex to genetically edit the T cell to produce gene edited T cells;
activating the gene edited T-cells within 24 hours of electroporation by CD3/CD28 microbead antigen-induced activation; and
transducing the genetically modified, activated T cells within 24 hours of activation with a lentivirus based vector comprising a heterologous nucleic acid molecule encoding a chimeric antigen receptor.

45. The method of claim 44, wherein the method further comprises culturing the transduced T cells for a period of time, such as 1-5 days.

46. The method of any one of claims 44-45, wherein the gene editing edits the CD5 locus to disrupt the CD5 locus, knock-out the CD5 gene, and/or reduce the expression of CD5.

47. A method of manufacturing a CAR-T cell comprising:
i) isolating unstimulated T cells from a subject;
ii) electroporating the unstimulated T cells with a gene editing complex to genetically edit the T cell to produce unstimulated, gene edited T cells;
iii) activating the unstimulated, gene edited T-cells within 24 hours of electroporation by CD3/CD28 microbead antigen-induced activation to produce activated, gene edited T cells;
iv) transducing the activated, gene edited T cells within 24 hours of activation with a lentivirus based vector comprising a heterologous nucleic acid molecule encoding a chimeric antigen receptor;
v) culturing the T cells for 1 to 7 days after transductions;
vi) harvesting the T cells 1 to 7 days after transduction; and
vii) optionally, freezing the harvested cells.

48. A CAR-T cell produced according to the method of any one of claims 1-47.

49. A method of treating a cancer patient with a CAR-T cell, the method comprising:
gene editing isolated unstimulated T cells obtained from a subject;
electroporating the T cells with a gene editing complex to genetically edit the T cell to produce gene edited T cells;
activating the gene edited T-cells within 24 hours of electroporation by CD3/CD28 microbead antigen-induced activation;
transducing the genetically modified, activated T cells within 24 hours of activation with a lentivirus based vector comprising a heterologous nucleic acid molecule encoding a chimeric antigen receptor; and
administering the transduced T cells to the patient.

50. A method of treating a cancer patient with a CAR-T cell, the method comprising:
i) isolating unstimulated T cells from a subject;
ii) electroporating the unstimulated T cells with a gene editing complex to genetically edit the T cell to produce unstimulated, gene edited T cells;
iii) activating the unstimulated, gene edited T-cells within 24 hours of electroporation by CD3/CD28 microbead antigen-induced activation to produce activated, gene edited T cells;
iv) transducing the activated, gene edited T cells within 24 hours of activation with a lentivirus based vector comprising a heterologous nucleic acid molecule encoding a chimeric antigen receptor;
v) culturing the T cells for 1 to 7 days after transductions;
vi) harvesting the T cells 1 to 7 days after transduction;
vii) optionally, freezing the harvested cells; and
viii) administering the harvested T cells to the patient.


SEQUENCE LISTING (18 sequences)

SEQ ID NO: 1 - protein, 5 aa, synthetic construct
NYDVH

SEQ ID NO: 2 - protein, 16 aa, synthetic construct
VIWNYGNTDYNAAFIS

SEQ ID NO: 3 - protein, 13 aa, synthetic construct
NHGDGYYNWYFDV

SEQ ID NO: 4 - protein, 11 aa, synthetic construct
KASQDINKYIA

SEQ ID NO: 5 - protein, 7 aa, synthetic construct
YTSTLQP

SEQ ID NO: 6 - protein, 8 aa, synthetic construct
LQYDNLWT

SEQ ID NO: 7 - protein, 121 aa, synthetic construct
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSS

SEQ ID NO: 8 - protein, 106 aa, synthetic construct
NIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIK

SEQ ID NO: 9 - protein, 242 aa, synthetic construct
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSGGGGSGGGGSGGGGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIK

SEQ ID NO: 10 - protein, 242 aa, synthetic construct
NIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKGGGGSGGGGSGGGGSEVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSS

SEQ ID NO: 11 - protein, 494 aa, synthetic construct
MALPVTALLLPLALLLHAARPGSEVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSGGGGSGGGGSGGGGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 471 aa, synthetic construct
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSGGGGSGGGGSGGGGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 5 aa, synthetic construct
GGGGS

SEQ ID NO: 14 - protein, 10 aa, synthetic construct
GGGGSGGGGS

SEQ ID NO: 15 - protein, 15 aa, synthetic construct
GGGGSGGGGSGGGGS

SEQ ID NO: 16 - protein, 20 aa, synthetic construct
GGGGSGGGGSGGGGSGGGGS

SEQ ID NO: 17 - protein, 4 aa, synthetic construct
  REPEAT 1..4: note = Sequence may be present n times, wherein n is 1, 2, 3, 4, 5, 6, 7, 8, 9, or 10
GGGS

SEQ ID NO: 18 - protein, 4 aa, synthetic construct
GGGS